SEQUENCE LISTING (3 sequences)

SEQ ID NO: 1 - protein, 925 aa, Homo sapiens
MERDGCAGGGSRGGEGGRAPREGPAGNGRDRGRSHAAEAPGDPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 2 - protein, 6 aa, Artificial Sequence
  Description of Artificial Sequence Synthetic 6xHis tag
HHHHHH

SEQ ID NO: 3 - protein, 6 aa, Homo sapiens
GSGFHG

(19) United States
(12) Patent Application Publication
(10) Pub. No.: US 2021/0023234 A1
(43) Pub. Date: Jan. 28, 2021

(54) ECTONUCLEOTIDE PYROPHOSPHATE-PHOSPHODIESTERASE (ENPP) CONJUGATES AND USES THEREOF

(71) Applicant: AbbVie Inc., North Chicago, IL (US)

(72) Inventors: William Michael GALLATIN, Kirkland, WA (US); Gregory N DIETSCH, Snohomish, WA (US); Joshua ODINGO, Everett, WA (US); Vincent FLORIO, Seattle, WA (US)

(21) Appl. No.: 17/043,082

(22) PCT Filed: Mar. 28, 2019
(86) PCT No.: PCT/US2019/024698

§ 371 Date: Sep. 29, 2020

Related U.S. Application Data
(60) Provisional application No. 62/651,013, filed on Mar. 30, 2018.

(51) Int. Cl. A61K 47/64; A61K 47/54

(52) U.S. Cl. CPC A61K 47/64; A61K 47/545

(57) ABSTRACT

Disclosed herein are ecto-nucleotide pyrophosphatase/phosphodiesterase (ENPP) complexes and synthetic molecules that interact with an ENPP protein. In some embodiments, also disclosed herein are modified ENPP polypeptides in complex with a synthetic molecule described herein.

### CROSS-REFERENCE

This patent application is a national stage application filed under 35 U.S.C. § 371. Of International Application No. PCT/US2019/024698, filed on Mar. 28, 2019, and claims the benefit of U.S. Provisional Application No. 62/651,013, filed Mar. 30, 2018 which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Mar. 12, 2018, is named 51184-707_201_SL.txt and is 8,505 bytes in size

### BACKGROUND OF THE DISCLOSURE

Cancer immunotherapy comprises the use of the patient's immune system to combat tumor cells. In some instances, cancer immunotherapy utilizes the presence of tumor antigens (e.g., tumor-specific antigens) to facilitate the recognition of the tumor cells by the immune system. In other instances, cancer immunotherapy utilizes immune system components such as lymphocytes and cytokines to coordinate a general immune response.

### SUMMARY OF THE DISCLOSURE

In some embodiments, disclosed herein are ecto-nucleotide pyrophosphatase/phosphodiesterase (ENPP) complexes and synthetic molecules that interact with an ENPP protein. In some embodiments, also disclosed herein are modified ENPP polypeptides in complex with a synthetic molecule described herein.

Disclosed herein, in certain embodiments, is an ecto-nucleotide pyrophosphatase/phosphodiesterase (ENPP) complex having a synthetic molecule in contact with at least one residue at an amino acid position corresponding to amino acid residues D218, T256, F257, N277, L290, K295, W322, P323, D326, Y340, Y371, D376, H380, D423, H424, or H535 as set forth in SEQ ID NO: 1, wherein the synthetic molecule is not a hydrolysis product of a nucleoside triphosphate. In some embodiments, the synthetic molecule has a structure represented by Formula (I), or a pharmaceutically acceptable salt, solvate, or stereoisomer thereof:

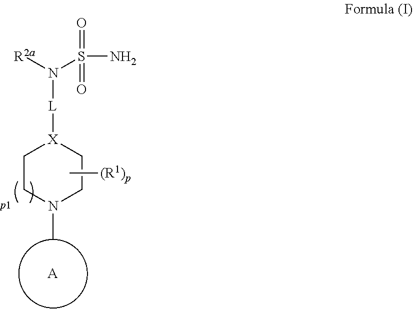

wherein
L is —(CR³R⁴)ₙ—;
X is —N— or —CH—;
Ring A is
   (a) an optionally substituted heteroaryl that is not quinazolinyl or pyrimidyl; or
   (b) an optionally substituted heterocycloalkyl; or
   (c) a ring selected from

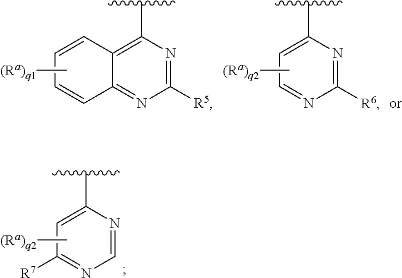

each R¹ is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, and optionally substituted (C₁-C₆ alkyl)heteroaryl;
or two R¹ on the same carbon are taken together to form an oxo;
R^{2a} is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R³ and R⁴ are independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R³ and R⁴ on the same carbon are taken together to form an oxo;
R⁵ is halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ heteroalkyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R⁶ is hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl; provided that R⁶ is not substituted imidazolyl;
R⁷ is hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted (C₁-C₆ alkyl)aryl, optionally substituted heteroaryl, or optionally substituted (C₁-C₆ alkyl)heteroaryl; provided that R⁷ is not substituted imidazolyl;
each R^{a} is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
n is 1-4;
p is 1-4;
p1 is 0 or 1;
q1 is 1-4; and
q2 is 1-2.

In some embodiments, the synthetic molecule has a structure represented by Formula (III), or a pharmaceutically acceptable salt, solvate, or stereoisomer thereof:

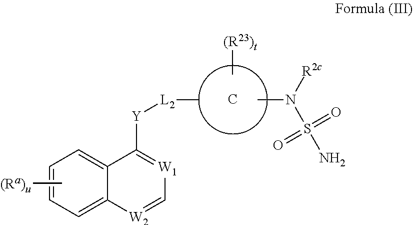

wherein
Y is —O— or —NR²⁰—;
L₂ is a bond or —(CR²¹R²²)ₙ₂—;
W₁ and W₂ are independently N or CR^{a}; provided that at least one of W₁ or W₂ is N;
Ring C is aryl, heteroaryl, cycloalkyl, or heterocycloalkyl;
each R²³ is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R^{2c} is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R²⁰ is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R²¹ and R²² are independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R²¹ and R²² on the same carbon are taken together to form an oxo;
each R^{a} is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
t is 1-4;
n2 is 1 or 2; and
u is 1-4.

In some embodiments, the molecule is in contact with at least one residue at an amino acid position corresponding to amino acid residues D218, T256, F257, N277, W322, D326, Y340, D376, H380, D423, H424, or H535 as set forth in SEQ ID NO: 1. In some embodiments, the molecule is in contact with at least one residue at an amino acid position corresponding to amino acid residues T256, F257, N277, W322, P323, D326, Y340, or Y371 as set forth in SEQ ID NO: 1. In some embodiments, the molecule is in contact with at least one residue at an amino acid position corresponding to amino acid residues T256, F257, N277, W322, P323, Y340, or Y371 as set forth in SEQ ID NO: 1. In some embodiments, the molecule is in contact with at least one residue at an amino acid position corresponding to amino acid residues F257, W322, D326, or Y340 as set forth in SEQ ID NO: 1. In some embodiments, the molecule is in contact with at least one residue at an amino acid position corresponding to amino acid residues F257 or Y340 as set forth in SEQ ID NO: 1. In some embodiments, the molecule is in contact with at least one residue at an amino acid position corresponding to amino acid residues D326 or W322 as set forth in SEQ ID NO: 1. In some embodiments, the molecule is in contact with a residue at an amino acid position corresponding to amino acid residue Y340 as set forth in SEQ ID NO: 1. In some embodiments, the molecule is in contact with a residue at an amino acid position corresponding to amino acid residue P323 as set forth in SEQ ID NO: 1. In some embodiments, the molecule is in contact with a residue at an amino acid position corresponding to amino acid residue W322 as set forth in SEQ ID NO: 1. In some embodiments, the molecule is in contact with a residue at an amino acid position corresponding to amino acid residue F257 as set forth in SEQ ID NO: 1. In some embodiments, the molecule is in contact with a residue at an amino acid position corresponding to amino acid residue N277 as set forth in SEQ ID NO: 1. In some embodiments, the molecule is in contact with a residue at an amino acid position corresponding to amino acid residue T256 as set forth in SEQ ID NO: 1. In some embodiments, the contact comprises covalent interaction, non-covalent interaction, or a combination thereof. In some embodiments, the contact comprises hydrogen bonding, hydrophobic interaction, ionic interaction, Van der Waals interaction, electrostatic interaction, pi bonding, or a combination thereof. In some embodiments, the ecto-nucleotide pyrophosphatase/phosphodiesterase is ecto-nucleotide pyrophosphatase/phosphodiesterase 1 (ENPP1). In some embodiments, the molecule is in contact with at least one of the following residues D218, T256, F257, N277, L290, K295, W322, P323, D326, Y340, Y371, D376, H380, D423, H424, or H535; wherein the amino acid positions are set forth in SEQ ID NO: 1. In some embodiments, the molecule is in contact with at least one of the following residues D218, T256, F257, N277, L290, K295, W322, P323, D326, Y340, Y371, D376, H380, D423, H424, or H535; wherein the amino acid positions are set forth in SEQ ID NO: 1. In some embodiments, the molecule is in contact with at least one of the following residues D218, T256, F257, N277, W322, D326, Y340, D376, H380, D423, H424, or H535; wherein the amino acid positions are set forth in SEQ ID NO: 1. In some embodiments, the molecule is in contact with at least one of the following residues T256, F257, N277, W322, P323, D326, Y340, or Y371; wherein the amino acid positions are set forth in SEQ ID NO: 1. In some embodiments, the molecule is in contact with at least one of the following residues T256, F257, N277, W322, P323, Y340, or Y371; wherein the amino acid positions are set forth in SEQ ID NO: 1. In some embodiments, the molecule is in contact with at least one of the following residues F257, W322, D326, or Y340; wherein the amino acid positions are set forth in SEQ ID NO: 1. In some embodiments, the molecule is in contact with at least one of the following residues F257 or Y340; wherein the amino acid positions are set forth in SEQ ID NO: 1. In some embodiments, the molecule is in contact with at least one of the following residues D326 or W322; wherein the amino acid positions are set forth in SEQ ID NO: 1. In some embodiments, the molecule is in contact with Y340, wherein the amino acid position is set forth in SEQ ID NO: 1. In some embodiments, the molecule is in contact with P323, wherein the amino acid position is set forth in SEQ ID NO: 1. In some embodiments, the molecule is in contact with W322, wherein the amino acid position is set forth in SEQ ID NO: 1. In some embodiments, the molecule is in contact with F257, wherein the amino acid position is set forth in SEQ ID NO: 1. In some embodiments, the molecule is in contact with N277, wherein the amino acid position is set forth in SEQ ID NO: 1. In some embodiments, the molecule is in contact with T256, wherein the amino acid position is set forth in SEQ ID NO: 1. In some embodiments, R^{2a} is hydrogen. In some embodiments, L is —(CR³R⁴)ₙ—; n is 2; and each R³ and R⁴ are independently hydrogen or halogen. In some embodiments, X is —CH—. In some embodiments, X is —N—. In some embodiments, p1 is 1. In some embodiments, each R¹ is independently hydrogen, halogen, or optionally substituted C₁-C₆ alkyl. In some embodiments, each R¹ is hydrogen. In some embodiments, Ring A is selected from:

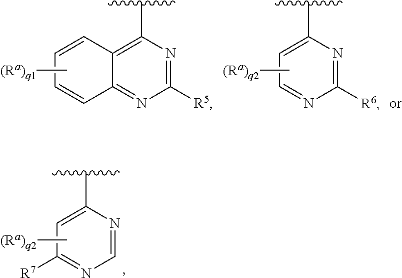

optionally substituted pyridinyl, optionally substituted pyrazinyl, optionally substituted pyridazinyl, optionally substituted pyrrolyl, optionally substituted pyrazolyl, optionally substituted imidazolyl, optionally substituted triazolyl, optionally substituted tetrazolyl, optionally substituted isoxazolyl, optionally substituted oxazolyl, optionally substituted isothiazolyl, optionally substituted thiazolyl, optionally substituted quinolinyl, optionally substituted isoquinolinyl, optionally substituted naphthyridinyl, optionally substituted cinnolinyl, optionally substituted pyridopyridazinyl, optionally substituted phthalazinyl, optionally substituted indolyl, optionally substituted pyrrolopyridinyl, optionally substituted indazolyl, optionally substituted pyrazolopyridine, optionally substituted benzotriazolyl, optionally substituted benzimidazolyl, optionally substituted pyrrolopyrimidinyl, optionally substituted pyrazolopyrimidinyl, optionally substituted triazolopyrimidinyl, optionally substituted purinyl, optionally substituted pyrrolopyridinyl, optionally substituted pyrazolopyridinyl, optionally substituted triazolopyridinyl, optionally substituted imidazopyridinyl, optionally substituted pyrrolo[2,1-f][1,2,4]triazinyl, optionally substituted pyrazolo[5,1-f][1,2,4]triazinyl, optionally substituted imidazo[5,1-f][1,2,4]triazinyl, optionally substituted imidazo[2,1-f][1,2,4]triazinyl, optionally substituted pyrrolo[1,2-a]pyrazinyl, optionally substituted pyrazolo[1,5-a]pyrazinyl, optionally substituted imidazo[1,5-a]pyrazinyl, optionally substituted imidazo[1,2-a]pyrazinyl, optionally substituted pyrrolo[1,2-c]pyrimidinyl, optionally substituted pyrazolo[1,5-c]pyrimidinyl, optionally substituted imidazo[1,5-c]pyrimidinyl, optionally substituted imidazo[1,2-c]pyrimidinyl, optionally substituted pyrrolo[1,2-b]pyridazinyl, optionally substituted pyrazolo[1,5-b]pyridazinyl, optionally substituted imidazo[1,5-b]pyridazinyl, optionally substituted imidazo[1,2-b]pyridazinyl, optionally substituted indolizinyl, optionally substituted pyrazolo[1,5-a]pyridinyl, optionally substituted imidazo[1,5-a]pyridinyl, optionally substituted imidazo[1,5-a]pyridinyl, optionally substituted imidazo[1,2-a]pyridinyl, optionally substituted pyrrolo[1,2-a][1,3,5]triazinyl, optionally substituted pyrazolo[1,5-a][1,3,5]triazinyl, optionally substituted imidazo[1,5-a][1,3,5]triazinyl, optionally substituted imidazo[1,2-a][1,3,5]triazinyl, optionally substituted pyrrolo[1,2-c]pyrimidinyl, optionally substituted pyrazolo[1,5-c]pyrimidinyl, optionally substituted imidazo[1,5-c]pyrimidinyl, optionally substituted imidazo[1,2-c]pyrimidinyl, optionally substituted pyrrolo[1,2-a]pyrazinyl, optionally substituted pyrazolo[1,5-a]pyrazinyl, optionally substituted imidazo[1,5-a]pyrazinyl, optionally substituted imidazo[1,2-a]pyrazinyl, optionally substituted pyrrolo[1,2-a]pyrimidinyl, optionally substituted pyrazolo[1,5-a]pyrimidinyl, optionally substituted imidazo[1,5-a]pyrimidinyl, optionally substituted imidazo[1,2-a]pyrimidinyl, optionally substituted tetrahydroquinazolinyl, optionally substituted dihydropyranopyrimidinyl, optionally substituted tetrahydropyridopyrimidinyl, optionally substituted tetrahydroquinolinyl, optionally substituted dihydropyranopyridinyl, optionally substituted tetrahydronaphthyridinyl, optionally substituted tetrahydroisoquinolinyl, optionally substituted dihydropyranopyridinyl, optionally substituted tetrahydronaphthyridinyl, optionally substituted dihydropurinone, optionally substituted dihydroimidazopyridinone, optionally substituted dihydrobenzoimidazolone, optionally substituted dihydropyrrolopyrimidinone, optionally substituted dihydropyrrolopyridinone, and optionally substituted indolinone.

In some embodiments, Ring A is selected from:

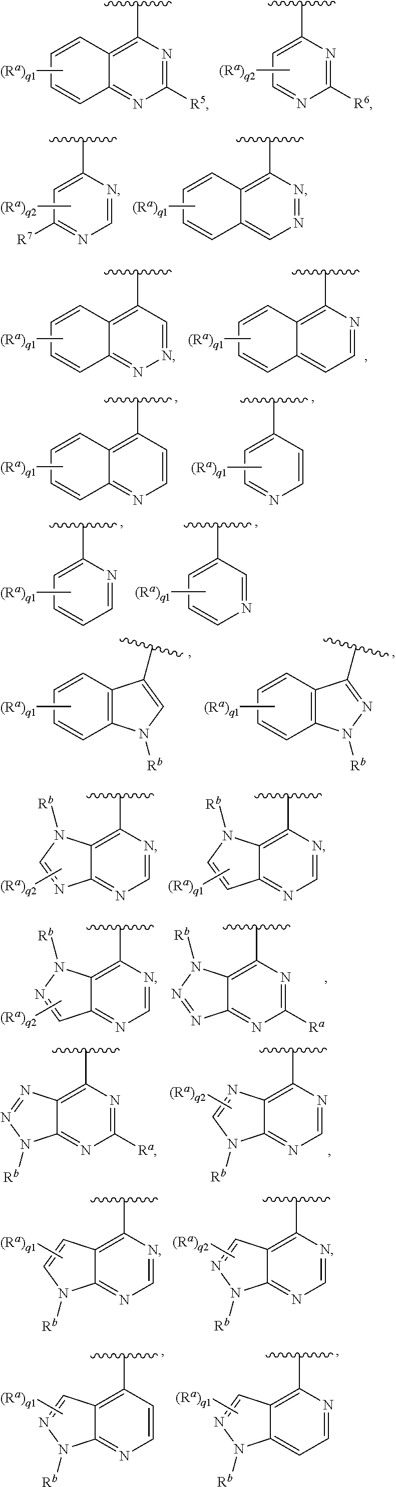

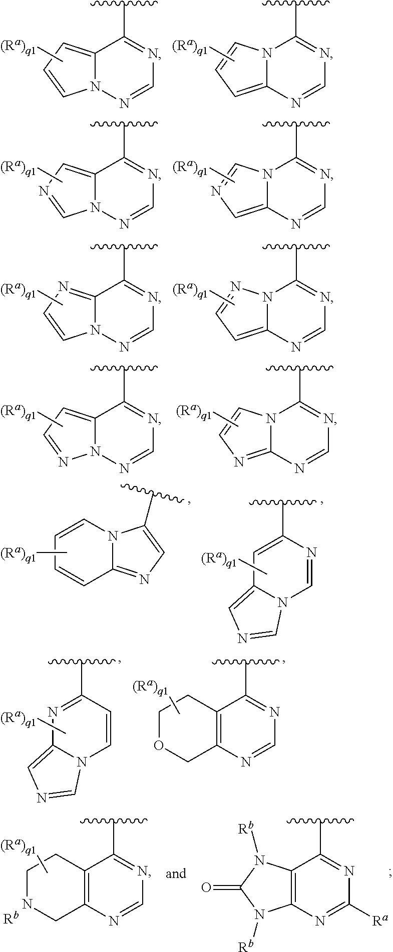

and

each R^{b} is independently hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl.

In some embodiments, Ring A is selected from:

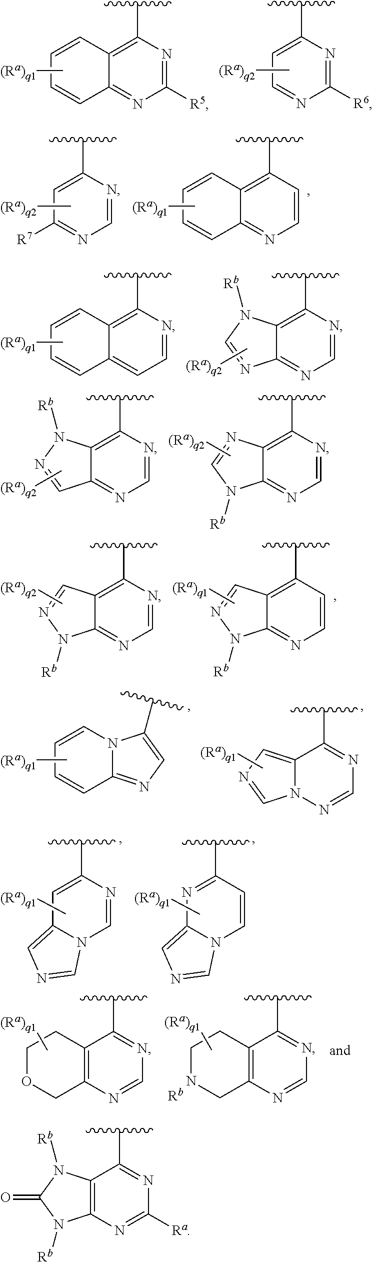

In some embodiments, Ring A is

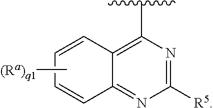

In some embodiments, each R^{a} is independently hydrogen, halogen, —CN, —OR¹¹, optionally substituted C₁-C₆ alkyl, or optionally substituted C₁-C₆ heteroalkyl; and q1 is 2 or 3. In some embodiments, each R^{a} is —OR¹¹; and q1 is 2. In some embodiments, R⁵ is halogen, —CN, —OR¹¹, —NR¹¹R¹², —C(═O)OR¹¹, —NR¹¹C(═O)R¹⁰, optionally substituted C₁-C₆ heteroalkyl, optionally substituted cycloalkyl, optionally substituted heterocycloalkyl, optionally substituted aryl, or optionally substituted heteroaryl. In some embodiments, R⁵ is —NR¹¹R¹², —NR¹¹C(═O)R¹⁰, optionally substituted aryl, or optionally substituted heteroaryl. In some embodiments, Ring A is

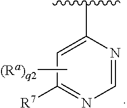

In some embodiments, each R^{a} is independently hydrogen, halogen, —CN, —OR¹¹, optionally substituted C₁-C₆ alkyl, or optionally substituted C₁-C₆ heteroalkyl; and q2 is 1. In some embodiments, R^{a} is hydrogen or C₁-C₆ alkyl; and q2 is 1. In some embodiments, R⁷ is hydrogen, halogen, —CN, —OR¹¹, —NR¹¹R¹², —NR¹¹C(═O)R¹⁰, —C(═O)NR¹¹R¹², optionally substituted C₁-C₆ alkyl, optionally substituted C₁-C₆ heteroalkyl, optionally substituted cycloalkyl, optionally substituted heterocycloalkyl, optionally substituted aryl, or optionally substituted heteroaryl; provided that R⁷ is not substituted imidazolyl. In some embodiments, R⁷ is optionally substituted C₁-C₆ alkyl, optionally substituted aryl, or optionally substituted heteroaryl; provided that R⁷ is not substituted imidazolyl. In some embodiments, R⁷ is optionally substituted C₁-C₆ alkyl or optionally substituted aryl. In some embodiments, Ring A is

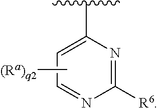

In some embodiments, each R^{a} is independently hydrogen, halogen, —CN, —OR¹¹, optionally substituted C₁-C₆ alkyl, or optionally substituted C₁-C₆ heteroalkyl; and q2 is 1. In some embodiments, each R^{a} is hydrogen. In some embodiments, R⁶ is hydrogen, halogen, —CN, —OR¹¹, —NR¹¹R¹², —NR¹¹C(═O)R¹⁰, optionally substituted C₁-C₆ alkyl, optionally substituted C₁-C₆ heteroalkyl, optionally substituted cycloalkyl, optionally substituted heterocycloalkyl, optionally substituted aryl, or optionally substituted heteroaryl; provided that R⁶ is not substituted imidazolyl. In some embodiments, R⁶ is hydrogen, —NR¹¹R¹², —NR¹¹C(═O)R¹⁰, or optionally substituted heteroaryl; provided that R⁶ is not substituted imidazolyl. In some embodiments, R⁶ is hydrogen, —NR¹¹R¹² or —NR¹¹C(═O)R¹⁰. In some embodiments, Ring A is selected from:

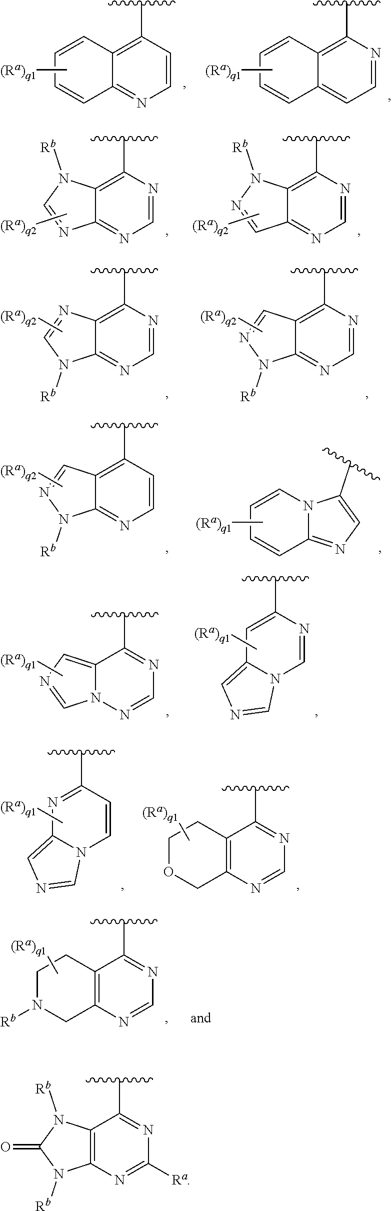

In some embodiments, each R^{a} is independently hydrogen, halogen, —CN, —OR¹¹, —NR¹¹R¹², —C(═O)OR¹¹, —OC(═O)OR¹¹, —C(═O)NR¹¹R¹², optionally substituted C₁-C₆ alkyl, optionally substituted C₁-C₆ heteroalkyl, optionally substituted cycloalkyl, or optionally substituted heterocycloalkyl; each R^{b} is independently hydrogen, optionally substituted C₁-C₆ alkyl, or optionally substituted aryl; q1 is 1 or 2; and q2 is 1. In some embodiments, R¹⁰ is optionally substituted C₁-C₆ alkyl, optionally substituted aryl, or optionally substituted heteroaryl. In some embodiments, each R¹¹ and R¹² are each independently hydrogen, optionally substituted C₁-C₆ alkyl, optionally substituted aryl, or optionally substituted heteroaryl. In some embodiments, each R¹¹ is C₁-C₆ alkyl. In some embodiments, W₁ and W₂ are N. In some embodiments, W₁ is N; and W₂ is CR^{a}. In some embodiments, W₁ is CR^{a}; and W₂ is N. In some embodiments, each R^{a} is independently hydrogen, halogen, —CN, —OR¹¹, —NR¹¹R¹², —C(═O)OR¹¹, —OC(═O)OR¹¹, —C(═O)NR¹¹R¹², optionally substituted C₁-C₆ alkyl, optionally substituted C₁-C₆ heteroalkyl, optionally substituted cycloalkyl, or optionally substituted heterocycloalkyl; and u is 1-3. In some embodiments, each R^{a} is —OR¹¹; and u is 1 or 2. In some embodiments, each R²³ is independently hydrogen, halogen, or optionally substituted C₁-C₆ alkyl. In some embodiments, each R²³ is hydrogen. In some embodiments, Y is —NR²⁰—. In some embodiments, Y is —O—. In some embodiments, R²⁰ is hydrogen or C₁-C₆ alkyl. In some embodiments, L₂ is a bond. In some embodiments, L₂ is —(CR²¹R²²)ₙ₂—; n2 is 1 or 2; and each R²¹ and R²² are independently hydrogen or halogen. In some embodiments, R^{2c} is hydrogen. In some embodiments, Ring C is a 6-membered aryl. In some embodiments, Ring C is a 5-membered heteroaryl. In some embodiments, Ring C is a 6-membered heteroaryl. In some embodiments, R¹⁰ is optionally substituted C₁-C₆ alkyl. In some embodiments, each R¹¹ and R¹² are each independently hydrogen or optionally substituted C₁-C₆ alkyl. In some embodiments, each R¹¹ is C₁-C₆ alkyl. In some embodiments, the hydrolysis product is AMP, TMP, GMP, or CMP.

Disclosed herein, in certain embodiments, is a synthetic molecule that is in contact with at least one residue at an amino acid position corresponding to amino acid residues D218, T256, F257, N277, L290, K295, W322, P323, D326, Y340, Y371, D376, H380, D423, H424, or H535 as set forth in SEQ ID NO: 1, wherein the synthetic molecule is not a hydrolysis product of a nucleoside triphosphate. In some embodiments, the synthetic molecule is in contact with at least one residue at an amino acid position corresponding to amino acid residues D218, T256, F257, N277, W322, D326, Y340, D376, H380, D423, H424, or H535 as set forth in SEQ ID NO: 1. In some embodiments, the synthetic molecule is in contact with at least one residue at an amino acid position corresponding to amino acid residues T256, F257, N277, W322, P323, D326, Y340, or Y371 as set forth in SEQ ID NO: 1. In some embodiments, the synthetic molecule is in contact with at least one residue at an amino acid position corresponding to amino acid residues T256, F257, N277, W322, P323, Y340, or Y371 as set forth in SEQ ID NO: 1. In some embodiments, the synthetic molecule is in contact with at least one residue at an amino acid position corresponding to amino acid residues F257, W322, D326, or Y340 as set forth in SEQ ID NO: 1. In some embodiments, the synthetic molecule is in contact with at least one residue at an amino acid position corresponding to amino acid residues F257 or Y340 as set forth in SEQ ID NO: 1. In some embodiments, the synthetic molecule is in contact with at least one residue at an amino acid position corresponding to amino acid residues D326 or W322 as set forth in SEQ ID NO: 1. In some embodiments, the synthetic molecule is in contact with a residue at an amino acid position corresponding to amino acid residue Y340 as set forth in SEQ ID NO: 1. In some embodiments, the synthetic molecule is in contact with a residue at an amino acid position corresponding to amino acid residue P323 as set forth in SEQ ID NO: 1. In some embodiments, the synthetic molecule is in contact with a residue at an amino acid position corresponding to amino acid residue W322 as set forth in SEQ ID NO: 1. In some embodiments, the synthetic molecule is in contact with a residue at an amino acid position corresponding to amino acid residue F257 as set forth in SEQ ID NO: 1. In some embodiments, the synthetic molecule is in contact with a residue at an amino acid position corresponding to amino acid residue N277 as set forth in SEQ ID NO: 1. In some embodiments, the synthetic molecule is in contact with a residue at an amino acid position corresponding to amino acid residue T256 as set forth in SEQ ID NO: 1. In some embodiments, the contact comprises hydrogen bonding, hydrophobic interaction, or ionic interaction. In some embodiments, the ecto-nucleotide pyrophosphatase/phosphodiesterase is ecto-nucleotide pyrophosphatase/phosphodiesterase 1 (ENPP1). In some embodiments, the synthetic molecule is in contact with at least one of the following residues D218, T256, F257, N277, L290, K295, W322, P323, D326, Y340, Y371, D376, H380, D423, H424, or H535; wherein the amino acid positions are set forth in SEQ ID NO: 1. In some embodiments, the synthetic molecule is in contact with at least one of the following residues D218, T256, F257, N277, L290, K295, W322, P323, D326, Y340, Y371, D376, H380, D423, H424, or H535; wherein the amino acid positions are set forth in SEQ ID NO: 1. In some embodiments, the synthetic molecule is in contact with at least one of the following residues D218, T256, F257, N277, W322, D326, Y340, D376, H380, D423, H424, or H535; wherein the amino acid positions are set forth in SEQ ID NO: 1. In some embodiments, the synthetic molecule is in contact with at least one of the following residues T256, F257, N277, W322, P323, D326, Y340, or Y371; wherein the amino acid positions are set forth in SEQ ID NO: 1. In some embodiments, the synthetic molecule is in contact with at least one of the following residues T256, F257, N277, W322, P323, Y340, or Y371; wherein the amino acid positions are set forth in SEQ ID NO: 1. In some embodiments, the synthetic molecule is in contact with at least one of the following residues F257, W322, D326, or Y340; wherein the amino acid positions are set forth in SEQ ID NO: 1. In some embodiments, the synthetic molecule is in contact with at least one of the following residues F257 or Y340; wherein the amino acid positions are set forth in SEQ ID NO: 1. In some embodiments, the synthetic molecule is in contact with at least one of the following residues D326 or W322; wherein the amino acid positions are set forth in SEQ ID NO: 1. In some embodiments, the synthetic molecule is in contact with Y340, wherein the amino acid position is set forth in SEQ ID NO: 1. In some embodiments, the synthetic molecule is in contact with P323, wherein the amino acid position is set forth in SEQ ID NO: 1. In some embodiments, the synthetic molecule is in contact with W322, wherein the amino acid position is set forth in SEQ ID NO: 1. In some embodiments, the synthetic molecule is in contact with F257, wherein the amino acid position is set forth in SEQ ID NO: 1. In some embodiments, the synthetic molecule is in contact with N277, wherein the amino acid position is set forth in SEQ ID NO: 1. In some embodiments, the synthetic molecule is in contact with T256, wherein the amino acid position is set forth in SEQ ID NO: 1. In some embodiments, the synthetic molecule has a structure represented by Formula (I), or a pharmaceutically acceptable salt, solvate, or stereoisomer thereof:

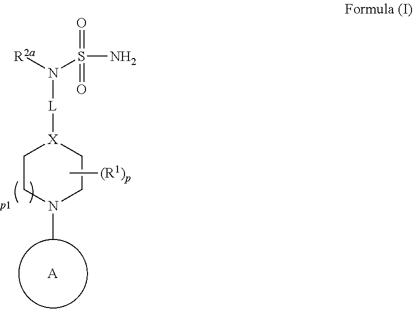

wherein
L is —(CR³R⁴)ₙ—;
X is —N— or —CH—;
Ring A is
   (a) an optionally substituted heteroaryl that is not quinazolinyl or pyrimidyl; or
   (b) an optionally substituted heterocycloalkyl; or
   (c) a ring selected from

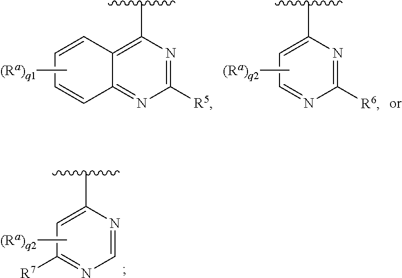

each R¹ is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, and optionally substituted (C₁-C₆ alkyl)heteroaryl;
or two R¹ on the same carbon are taken together to form an oxo;
R^{2a} is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R³ and R⁴ are independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R³ and R⁴ on the same carbon are taken together to form an oxo;
R⁵ is halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ heteroalkyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R⁶ is hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl; provided that R⁶ is not substituted imidazolyl;
R⁷ is hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted (C₁-C₆ alkyl)aryl, optionally substituted heteroaryl, or optionally substituted (C₁-C₆ alkyl)heteroaryl; provided that R⁷ is not substituted imidazolyl;
each R^{a} is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
n is 1-4;
p is 1-4;
p1 is 0 or 1;
q1 is 1-4; and
q2 is 1-2.

In some embodiments, the synthetic molecule has a structure represented by Formula (III), or a pharmaceutically acceptable salt, solvate, or stereoisomer thereof:

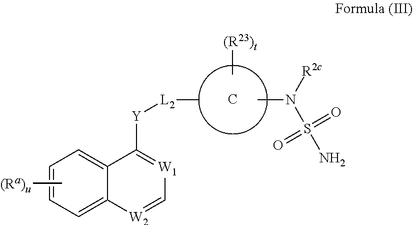

wherein
Y is —O— or —NR²⁰—;
L₂ is a bond or —(CR²¹R²²)ₙ₂—;
W₁ and W₂ are independently N or CR^{a}; provided that at least one of W₁ or W₂ is N;
Ring C is aryl, heteroaryl, cycloalkyl, or heterocycloalkyl;
each R²³ is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R^{2c} is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R²⁰ is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R²¹ and R²² are independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R²¹ and R²² on the same carbon are taken together to form an oxo;
each R^{a} is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
t is 1-4;
n2 is 1 or 2; and
u is 1-4.

In some embodiments, the hydrolysis product is AMP, TMP, GMP, or CMP.

Disclosed herein, in certain embodiments, is a modified ecto-nucleotide pyrophosphatase/phosphodiesterase (ENPP) polypeptide comprising a synthetic molecule that is in contact with at least one residue at an amino acid position corresponding to amino acid residues D218, T256, F257, N277, L290, K295, W322, P323, D326, Y340, Y371, D376, H380, D423, H424, or H535 as set forth in SEQ ID NO: 1; wherein the synthetic molecule competes with 2-(1-(6,7-Dimethoxyquinazolin-4-yl)piperidin-4-yl)ethyl sulfamide for contact with ENPP, and wherein the synthetic molecule is not a hydrolysis product of a nucleoside triphosphate. In some embodiments, the synthetic molecule is in contact with at least one residue at an amino acid position corresponding to amino acid residues D218, T256, F257, N277, W322, D326, Y340, D376, H380, D423, H424, or H535 as set forth in SEQ ID NO: 1. In some embodiments, the synthetic molecule is in contact with at least one residue at an amino acid position corresponding to amino acid residues T256, F257, N277, W322, P323, D326, Y340, or Y371 as set forth in SEQ ID NO: 1. In some embodiments, the synthetic molecule is in contact with at least one residue at an amino acid position corresponding to amino acid residues T256, F257, N277, W322, P323, Y340, or Y371 as set forth in SEQ ID NO: 1. In some embodiments, the synthetic molecule is in contact with at least one residue at an amino acid position corresponding to amino acid residues F257, W322, D326, or Y340 as set forth in SEQ ID NO: 1. In some embodiments, the synthetic molecule is in contact with at least one residue at an amino acid position corresponding to amino acid residues F257 or Y340 as set forth in SEQ ID NO: 1. In some embodiments, the synthetic molecule is in contact with at least one residue at an amino acid position corresponding to amino acid residues D326 or W322 as set forth in SEQ ID NO: 1. In some embodiments, the synthetic molecule is in contact with a residue at an amino acid position corresponding to amino acid residue Y340 as set forth in SEQ ID NO: 1. In some embodiments, the synthetic molecule is in contact with a residue at an amino acid position corresponding to amino acid residue P323 as set forth in SEQ ID NO: 1. In some embodiments, the synthetic molecule is in contact with a residue at an amino acid position corresponding to amino acid residue W322 as set forth in SEQ ID NO: 1. In some embodiments, the synthetic molecule is in contact with a residue at an amino acid position corresponding to amino acid residue F257 as set forth in SEQ ID NO: 1. In some embodiments, the synthetic molecule is in contact with a residue at an amino acid position corresponding to amino acid residue N277 as set forth in SEQ ID NO: 1. In some embodiments, the synthetic molecule is in contact with a residue at an amino acid position corresponding to amino acid residue T256 as set forth in SEQ ID NO: 1. In some embodiments, the contact comprises hydrogen bonding, hydrophobic interaction, or ionic interaction. In some embodiments, the ecto-nucleotide pyrophosphatase/phosphodiesterase is ecto-nucleotide pyrophosphatase/phosphodiesterase 1 (ENPP1). In some embodiments, the synthetic molecule is in contact with at least one of the following residues D218, T256, F257, N277, L290, K295, W322, P323, D326, Y340, Y371, D376, H380, D423, H424, or H535; wherein the amino acid positions are set forth in SEQ ID NO: 1. In some embodiments, the synthetic molecule is in contact with at least one of the following residues D218, T256, F257, N277, L290, K295, W322, P323, D326, Y340, Y371, D376, H380, D423, H424, or H535; wherein the amino acid positions are set forth in SEQ ID NO: 1. In some embodiments, the synthetic molecule is in contact with at least one of the following residues D218, T256, F257, N277, W322, D326, Y340, D376, H380, D423, H424, or H535; wherein the amino acid positions are set forth in SEQ ID NO: 1. In some embodiments, the synthetic molecule is in contact with at least one of the following residues T256, F257, N277, W322, P323, D326, Y340, or Y371; wherein the amino acid positions are set forth in SEQ ID NO: 1. In some embodiments, the synthetic molecule is in contact with at least one of the following residues T256, F257, N277, W322, P323, Y340, or Y371; wherein the amino acid positions are set forth in SEQ ID NO: 1. In some embodiments, the synthetic molecule is in contact with at least one of the following residues F257, W322, D326, or Y340; wherein the amino acid positions are set forth in SEQ ID NO: 1. In some embodiments, the synthetic molecule is in contact with at least one of the following residues F257 or Y340; wherein the amino acid positions are set forth in SEQ ID NO: 1. In some embodiments, the synthetic molecule is in contact with at least one of the following residues D326 or W322; wherein the amino acid positions are set forth in SEQ ID NO: 1. In some embodiments, the synthetic molecule is in contact with Y340, wherein the amino acid position is set forth in SEQ ID NO: 1. In some embodiments, the synthetic molecule is in contact with P323, wherein the amino acid position is set forth in SEQ ID NO: 1. In some embodiments, the synthetic molecule is in contact with W322, wherein the amino acid position is set forth in SEQ ID NO: 1. In some embodiments, the synthetic molecule is in contact with F257, wherein the amino acid position is set forth in SEQ ID NO: 1. In some embodiments, the synthetic molecule is in contact with N277, wherein the amino acid position is set forth in SEQ ID NO: 1. In some embodiments, the synthetic molecule is in contact with T256, wherein the amino acid position is set forth in SEQ ID NO: 1. In some embodiments, the synthetic molecule has a structure represented by Formula (I), or a pharmaceutically acceptable salt, solvate, or stereoisomer thereof:

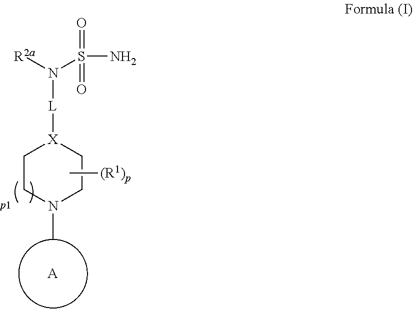

wherein
L is —(CR³R⁴)ₙ—;
X is —N— or —CH—;
Ring A is
   (d) an optionally substituted heteroaryl that is not quinazolinyl or pyrimidyl; or
   (e) an optionally substituted heterocycloalkyl; or
   (f) a ring selected from

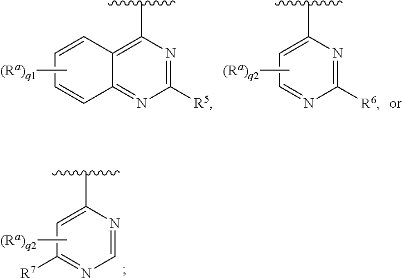

each R¹ is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, and optionally substituted (C₁-C₆ alkyl)heteroaryl;
or two R¹ on the same carbon are taken together to form an oxo;
R^{2a} is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R³ and R⁴ are independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R³ and R⁴ on the same carbon are taken together to form an oxo;
R⁵ is halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ heteroalkyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R⁶ is hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl; provided that R⁶ is not substituted imidazolyl;
R⁷ is hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted (C₁-C₆ alkyl)aryl, optionally substituted heteroaryl, or optionally substituted (C₁-C₆ alkyl)heteroaryl; provided that R⁷ is not substituted imidazolyl;
each R^{a} is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
n is 1-4;
p is 1-4;
p1 is 0 or 1;
q1 is 1-4; and
q2 is 1-2.

In some embodiments, the synthetic molecule has a structure represented by Formula (III), or a pharmaceutically acceptable salt, solvate, or stereoisomer thereof:

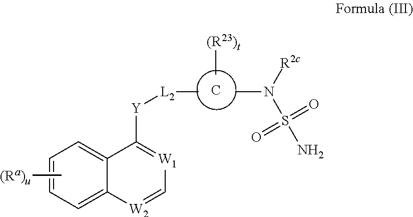

wherein
Y is —O— or —NR²⁰—;
L₂ is a bond or —(CR²¹R²²)ₙ₂—;
W₁ and W₂ are independently N or CR^{a}; provided that at least one of W₁ or W₂ is N;
Ring C is aryl, heteroaryl, cycloalkyl, or heterocycloalkyl;
each R²³ is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R^{2c} is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R²⁰ is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R²¹ and R²² are independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R²¹ and R²² on the same carbon are taken together to form an oxo;
each R^{a} is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
t is 1-4;
n2 is 1 or 2; and
u is 1-4.

### BRIEF DESCRIPTION OF THE DRAWINGS

Various aspects of the disclosure are set forth with particularity in the appended claims. The patent application file contains at least one drawing executed in color. Copies of this patent application with color drawing(s) will be provided by the Office upon request and payment of the necessary fee. A better understanding of the features and advantages of the present disclosure will be obtained by reference to the following detailed description that sets forth illustrative embodiments, in which the principles of the disclosure are utilized, and the accompanying drawings of which:

FIG. 1 illustrates an exemplary chimeric human ENPP1 (hENPP1) construct. Residues Asn54, Asn285, Asn341, Asn477, Asn585, and Asn807 are the respective N-linked glycosylation sites in hENPP1 and hENPP2. Figure discloses “6x-His” as SEQ ID NO: 2.

FIG. 2 illustrates the crystal structure of the hENPP1 in complex with Compound 3. The catalytic domain of hENPP1 is shown in green.

FIG. 3 illustrates an exemplary close-up view of Compound 3 within the interaction pocket. Figure discloses “GSGFHG” as SEQ ID NO: 3.

FIG. 4 illustrates an exemplary close-up view of Compound 3 within the interaction pocket. Human ENPP1 is shown as an electrostatic potential surface model.

### DETAILED DESCRIPTION OF THE DISCLOSURE

Cytosolic DNA signals the presence of cellular damage and/or the presence of cancerous cells. These cytosolic DNAs (e.g., double stranded DNAs) are surveyed by DNA sensors such as RNA pol III, DAI, IFI16, DDX41, LSm14A, cyclic-GMP-AMP synthase, LRRFIP1, Sox2, DHX9/36, Ku70 and AIM2. Cyclic-GMP-AMP synthase (cGAS or cGAMP synthase) is a 522 amino acid protein that belongs to the nucleotidyltransferase family of cytosolic DNA sensors. Upon cytosolic DNA stimulation, cGAS synthesizes cGAMP, which comprises a first bond between the 2′-OH of GMP and the 5′-phosphate of AMP and a second bond between the 3′-OH of AMP and the 5′-phosphate of GMP. cGAMP (also known as cyclic GMP-AMP, 2′3′-cGAMP, cGAMP (2′-5′) or cyclic Gp(2′-5′)Ap(3′-5′)) serves as a ligand to STING, thereby activating the STING-mediated IFN (e.g., IFNβ) production.

STING (also known as stimulator of interferon genes, TMEM173, MITA, ERIS, or MPYS) is a 378 amino acid protein that comprises a N-terminal region containing four trans-membrane domains and a C-terminal domain that comprises a dimerization domain. Upon binding to 2′3′-cGAMP, STING undergoes a conformational rearrangement enclosing the 2′3′-cGAMP molecule.

Binding of 2′3′-cGAMP activates a cascade of events whereby STING recruits and activates IκB kinase (IKK) and TANK-binding kinase (TBK1), which following their phosphorylation, respectively activate nuclear transcription factor κB (NF-κB) and interferon regulatory factor 3 (IRF3). In some instances, the activated proteins translocate to the nucleus to induce transcription of the genes encoding type I IFN and cytokines for promoting intercellular host immune defense. In some cases, the production of type I IFNs further drives the development of cytolytic T cell response and enhances expression of MHC, thereby increasing antigen processing and presentation within a tumor microenvironment. In such cases, enhanced type I IFN production further renders the tumor cells to be more vulnerable by enhancing their recognition by the immune system.

In some embodiments, tumor cells circumvent the STING-mediated type I IFN production through overexpression of a phosphodiesterase. Phosphodiesterases comprise a class of enzymes that catalyze the hydrolysis of a phosphodiester bond. In some instances, this class comprises cyclic nucleotide phosphodiesterases, phospholipases C and D, autotaxin, sphingomyelin phosphodiesterase, DNases, RNases, restriction endonucleases, and small-molecule phosphodiesterases. In additional embodiments, the class of phosphodiesterases comprises an ecto-nucleotide pyrophosphatase/phosphodiesterase (ENPP).

Cyclic nucleotide phosphodiesterases (PDEs) and ENPP regulate the cyclic nucleotides cAMP and cGMP. In some instances, cAMP and cGMP function as intracellular second messengers to transduce a variety of extracellular signals including hormones, light, and neurotransmitters. In some cases, PDEs and ENPP degrade cyclic nucleotides to their corresponding monophosphates, thereby regulating the intracellular concentrations of cyclic nucleotides and their effects on signal transduction.

In some embodiments, disclosed herein are ecto-nucleotide pyrophosphatase/phosphodiesterase (ENPP) complexes and synthetic molecules that interact with an ENPP protein. In some embodiments, also disclosed herein are modified ENPP polypeptides in complex with a synthetic molecule described herein.

### Ecto-Nucleotide Pyrophosphatase/Phosphodiesterase

Ecto-nucleotide pyrophosphatase/phosphodiesterases (ENPP) or nucleotide pyrophosphatase/phosphodiesterases (NPP) are a subfamily of ectonucleotidases which hydrolyze the pyrophosphate and phosphodiester bonds of their substrates to nucleoside 5′-monophosphates. In some embodiments, ENPP (or NPP) comprises seven members, ENPP-1, ENPP-2, ENPP-3, ENPP-4, ENPP-5, ENPP-6, and ENPP-7.

The ecto-nucleotide pyrophosphatase/phosphodiesterase 1 (ENPP1) protein (also known as PC-1) is a type II transmembrane glycoprotein comprising two identical disulfide-bonded subunits. In some instances, ENPP1 is expressed in precursor cells and promotes osteoblast differentiation and regulates bone mineralization. In some instances, ENPP1 negatively regulates bone mineralization by hydrolyzing extracellular nucleotide triphosphates (NTPs) to produce inorganic pyrophosphate (PPi). In some cases, expression of ENPP1 has been observed in pancreas, kidney, bladder, and the liver. In some cases, ENPP-1 has been observed to be overexpressed in cancer cells, e.g., in breast cancer cells and glioblastoma cells.

In some embodiments, ENPP1 has a broad specificity and cleaves a variety of substrates, including phosphodiester bonds of nucleotides and nucleotide sugars and pyrophosphate bonds of nucleotides and nucleotide sugars. In some instances, ENPP1 functions to hydrolyze nucleoside 5′ triphosphates to their corresponding monophosphates and also hydrolyze diadenosine polyphosphates. In some cases, ENPP1 hydrolyzes the 2′5′ linkage of cyclic nucleotides. In some cases, ENPP1 degrades 2′3′-cGAMP, a substrate of STING.

In some embodiments, ENPP1 comprises two N-terminal somatomedin B (SMB)-like domains (SMB1 and SMB2), a catalytic domain and a C-terminal nuclease-like domain. In some cases, the two SMB domains is connected to the catalytic domain by a first flexible linker, while the catalytic domain is further connected to the nuclease-like domain by a second flexible linker. In some instances, the SMB domains facilitate ENPP1 dimerization. In some cases, the catalytic domain comprises the NTP binding site. In some cases, the nuclease-like domain comprises an EF-hand motif, which binds Ca⁺² ion.

In human ENPP1 (hENPP1), the catalytic domain comprises amino acid residues 191-591, in which the numbering corresponds to residues 191-591 as set forth in SEQ ID NO: 1. Residues Asn285, Asn341, Asn477, and Asn585 comprise the N-linked glycosylation site.

In some cases, ENPP2 and ENPP3 are type II transmembrane glycoproteins that share a similar architecture with ENPP1, for example, comprising the two N-terminal SMB-like domains, a catalytic domain, and a nuclease-like domain. In some instances, ENPP2 hydrolyzes lysophospholipids to produce lysophosphatidic acid (LPA) or sphingosylphosphorylcholine (SPC) to produce sphingosine-1 phosphate (S1P). In some cases, ENPP-3 is identified to regulate N-acetylglucosaminyltransferase GnT-IX (GnT-Vb).

In some embodiments, ENPP4-ENPP7 are shorter proteins compared to ENPP1-ENPP3 and comprise a catalytic domain and lack the SMB-like and nuclease-like domains. ENPP6 is a choline-specific glycerophosphodiesterase, with lysophospholipase C activity towards lysophosphatidylcholine (LPC). ENPP7 is an alkaline sphingomyelinase (alk-SMase) with no detectable nucleotidase activity.

### ENPP Complexes

In some embodiments, disclosed herein include an ecto-nucleotide pyrophosphatase/phosphodiesterase (ENPP) complex. In some instances, the synthetic compound is in contact with at least one residue at an amino acid position corresponding to amino acid residues 218, 256, 257, 277, 290, 295, 322, 323, 326, 340, 371, 376, 380, 423, 424, or 535 as set forth in SEQ ID NO: 1. In some instances, the synthetic compound is in contact with at least one residue at an amino acid position corresponding to amino acid residues 218, 256, 257, 277, 322, 326, 340, 376, 380, 423, 424, or 535 as set forth in SEQ ID NO: 1. In some instances, the synthetic molecule is in contact with at least one residue at an amino acid position corresponding to amino acid residues 256, 257, 277, 322, 323, 326, 340, or 371 as set forth in SEQ ID NO: 1. In some instances, the synthetic molecule is in contact with at least one residue at an amino acid position corresponding to amino acid residues 256, 257, 277, 322, 323, 340, or 371 as set forth in SEQ ID NO: 1. In some instances, the synthetic molecule is in contact with at least one residue at an amino acid position corresponding to amino acid residues 257, 322, 326, or 340 as set forth in SEQ ID NO: 1. In some instances, the synthetic molecule is in contact with at least one residue at an amino acid position corresponding to amino acid residues 257 or 340 as set forth in SEQ ID NO: 1. In some instances, the synthetic molecule is in contact with at least one residue at an amino acid position corresponding to amino acid residues 326 or 322 as set forth in SEQ ID NO: 1. In some instances, the synthetic molecule is in contact with a residue at an amino acid position corresponding to amino acid residue 340 as set forth in SEQ ID NO: 1. In some instances, the synthetic molecule is in contact with a residue at an amino acid position corresponding to amino acid residue 323 as set forth in SEQ ID NO: 1. In some instances, the synthetic molecule is in contact with a residue at an amino acid position corresponding to amino acid residue 322 as set forth in SEQ ID NO: 1. In some instances, the synthetic molecule is in contact with a residue at an amino acid position corresponding to amino acid residue 257 as set forth in SEQ ID NO: 1. In some instances, the synthetic molecule is in contact with a residue at an amino acid position corresponding to amino acid residue 277 as set forth in SEQ ID NO: 1. In some instances, the synthetic molecule is in contact with a residue at an amino acid position corresponding to amino acid residue 256 as set forth in SEQ ID NO: 1.

In some instances, the ENPP polypeptide is an ENPP1 polypeptide. In some cases, the synthetic molecule is in contact with at least one residue of ENPP1 at an amino acid position corresponding to amino acid residues 218, 256, 257, 277, 290, 295, 322, 323, 326, 340, 371, 376, 380, 423, 424, or 535 as set forth in SEQ ID NO: 1. In some instances, the synthetic molecule is in contact with at least one residue of ENPP1 at an amino acid position corresponding to amino acid residues 218, 256, 257, 277, 322, 326, 340, 376, 380, 423, 424, or 535 as set forth in SEQ ID NO: 1. In some instances, the synthetic molecule is in contact with at least one residue of ENPP1 at an amino acid position corresponding to amino acid residues 256, 257, 277, 322, 323, 326, 340, or 371 as set forth in SEQ ID NO: 1. In some instances, the synthetic molecule is in contact with at least one residue of ENPP1 at an amino acid position corresponding to amino acid residues 256, 257, 277, 322, 323, 340, or 371 as set forth in SEQ ID NO: 1. In some instances, the synthetic molecule is in contact with at least one residue of ENPP1 at an amino acid position corresponding to amino acid residues 257, 322, 326, or 340 as set forth in SEQ ID NO: 1. In some instances, the synthetic molecule is in contact with at least one residue of ENPP1 at an amino acid position corresponding to amino acid residues 257 or 340 as set forth in SEQ ID NO: 1. In some instances, the synthetic molecule is in contact with at least one residue of ENPP1 at an amino acid position corresponding to amino acid residues 326 or 322 as set forth in SEQ ID NO: 1. In some instances, the synthetic molecule is in contact with a residue of ENPP1 at an amino acid position corresponding to amino acid residue 340 as set forth in SEQ ID NO: 1. In some instances, the synthetic molecule is in contact with a residue at an amino acid position corresponding to amino acid residue 323 as set forth in SEQ ID NO: 1. In some instances, the synthetic molecule is in contact with a residue of ENPP1 at an amino acid position corresponding to amino acid residue 322 as set forth in SEQ ID NO: 1. In some instances, the synthetic molecule is in contact with a residue of ENPP1 at an amino acid position corresponding to amino acid residue 257 as set forth in SEQ ID NO: 1. In some instances, the synthetic molecule is in contact with a residue of ENPP1 at an amino acid position corresponding to amino acid residue 277 as set forth in SEQ ID NO: 1. In some instances, the synthetic molecule is in contact with a residue of ENPP1 at an amino acid position corresponding to amino acid residue 256 as set forth in SEQ ID NO: 1.

In some embodiments, the synthetic molecule is in contact with at least one residue at an amino acid position corresponding to amino acid residues D218, T256, F257, N277, L290, K295, W322, P323, D326, Y340, Y371, D376, H380, D423, H424, or H535 as set forth in SEQ ID NO: 1. In some instances, the synthetic molecule is in contact with at least one residue at an amino acid position corresponding to amino acid residues D218, T256, F257, N277, W322, D326, Y340, D376, H380, D423, H424, or H535 as set forth in SEQ ID NO: 1. In some instances, the synthetic molecule is in contact with at least one residue at an amino acid position corresponding to amino acid residues T256, F257, N277, W322, P323, D326, Y340, or Y371 as set forth in SEQ ID NO: 1. In some instances, the synthetic molecule is in contact with at least one residue at an amino acid position corresponding to amino acid residues T256, F257, N277, W322, P323, Y340, or Y371 as set forth in SEQ ID NO: 1. In some instances, the synthetic molecule is in contact with at least one residue at an amino acid position corresponding to amino acid residues F257, W322, D326, or Y340 as set forth in SEQ ID NO: 1. In some instances, the synthetic molecule is in contact with at least one residue at an amino acid position corresponding to amino acid residues F257 or Y340 as set forth in SEQ ID NO: 1. In some instances, the synthetic molecule is in contact with at least one residue at an amino acid position corresponding to amino acid residues D326 or W322 as set forth in SEQ ID NO: 1. In some instances, the synthetic molecule is in contact with a residue at an amino acid position corresponding to amino acid residue Y340 as set forth in SEQ ID NO: 1. In some instances, the synthetic molecule is in contact with a residue at an amino acid position corresponding to amino acid residue P323 as set forth in SEQ ID NO: 1. In some instances, the synthetic molecule is in contact with a residue at an amino acid position corresponding to amino acid residue W322 as set forth in SEQ ID NO: 1. In some instances, the synthetic molecule is in contact with a residue at an amino acid position corresponding to amino acid residue F257 as set forth in SEQ ID NO: 1. In some instances, the synthetic molecule is in contact with a residue at an amino acid position corresponding to amino acid residue N277 as set forth in SEQ ID NO: 1. In some instances, the synthetic molecule is in contact with a residue at an amino acid position corresponding to amino acid residue T256 as set forth in SEQ ID NO: 1.

In some instances, the ENPP polypeptide is an ENPP1 polypeptide. In some embodiments, the synthetic molecule is in contact with at least one residue of ENPP1 at an amino acid position corresponding to amino acid residues D218, T256, F257, N277, L290, K295, W322, P323, D326, Y340, Y371, D376, H380, D423, H424, or H535 as set forth in SEQ ID NO: 1. In some instances, the synthetic molecule is in contact with at least one residue of ENPP1 at an amino acid position corresponding to amino acid residues D218, T256, F257, N277, W322, D326, Y340, D376, H380, D423, H424, or H535 as set forth in SEQ ID NO: 1. In some instances, the synthetic molecule is in contact with at least one residue of ENPP1 at an amino acid position corresponding to amino acid residues T256, F257, N277, W322, P323, D326, Y340, or Y371 as set forth in SEQ ID NO: 1. In some instances, the synthetic molecule is in contact with at least one residue of ENPP1 at an amino acid position corresponding to amino acid residues T256, F257, N277, W322, P323, Y340, or Y371 as set forth in SEQ ID NO: 1. In some instances, the synthetic molecule is in contact with at least one residue of ENPP1 at an amino acid position corresponding to amino acid residues F257, W322, D326, or Y340 as set forth in SEQ ID NO: 1. In some instances, the synthetic molecule is in contact with at least one residue of ENPP1 at an amino acid position corresponding to amino acid residues F257 or Y340 as set forth in SEQ ID NO: 1. In some instances, the synthetic molecule is in contact with at least one residue of ENPP1 at an amino acid position corresponding to amino acid residues D326 or W322 as set forth in SEQ ID NO: 1. In some instances, the synthetic molecule is in contact with a residue of ENPP1 at an amino acid position corresponding to amino acid residue Y340 as set forth in SEQ ID NO: 1. In some instances, the synthetic molecule is in contact with a residue of ENPP1 at an amino acid position corresponding to amino acid residue P323 as set forth in SEQ ID NO: 1. In some instances, the synthetic molecule is in contact with a residue of ENPP1 at an amino acid position corresponding to amino acid residue W322 as set forth in SEQ ID NO: 1. In some instances, the synthetic molecule is in contact with a residue of ENPP1 at an amino acid position corresponding to amino acid residue F257 as set forth in SEQ ID NO: 1. In some instances, the synthetic molecule is in contact with a residue of ENPP1 at an amino acid position corresponding to amino acid residue N277 as set forth in SEQ ID NO: 1. In some instances, the synthetic molecule is in contact with a residue of ENPP1 at an amino acid position corresponding to amino acid residue T256 as set forth in SEQ ID NO: 1. In some instances, the ENPP1 polypeptide is a human ENPP1 polypeptide.

In some embodiments, also described herein is a modified ecto-nucleotide pyrophosphatase/phosphodiesterase (ENPP) polypeptide comprising a synthetic molecule that is in contact with at least one residue at an amino acid position corresponding to amino acid residues 218, 256, 257, 277, 290, 295, 322, 323, 326, 340, 371, 376, 380, 423, 424, or 535 as set forth in SEQ ID NO: 1; in which the synthetic molecule competes with 2-(1-(6,7-Dimethoxyquinazolin-4-yl)piperidin-4-yl)ethyl sulfamide for contact with ENPP. In some instances, the synthetic molecule is in contact with at least one residue at an amino acid position corresponding to amino acid residues 218, 256, 257, 277, 322, 326, 340, 376, 380, 423, 424, or 535 as set forth in SEQ ID NO: 1. In some instances, the synthetic molecule is in contact with at least one residue at an amino acid position corresponding to amino acid residues 256, 257, 277, 322, 323, 326, 340, or 371 as set forth in SEQ ID NO: 1. In some instances, the synthetic molecule is in contact with at least one residue at an amino acid position corresponding to amino acid residues 256, 257, 277, 322, 323, 340, or 371 as set forth in SEQ ID NO: 1. In some instances, the synthetic molecule is in contact with at least one residue at an amino acid position corresponding to amino acid residues 257, 322, 326, or 340 as set forth in SEQ ID NO: 1. In some instances, the synthetic molecule is in contact with at least one residue at an amino acid position corresponding to amino acid residues 257 or 340 as set forth in SEQ ID NO: 1. In some instances, the synthetic molecule is in contact with at least one residue at an amino acid position corresponding to amino acid residues 326 or 322 as set forth in SEQ ID NO: 1. In some instances, the synthetic molecule is in contact with a residue at an amino acid position corresponding to amino acid residue 340 as set forth in SEQ ID NO: 1. In some instances, the synthetic molecule is in contact with a residue at an amino acid position corresponding to amino acid residue 323 as set forth in SEQ ID NO: 1. In some instances, the synthetic molecule is in contact with a residue at an amino acid position corresponding to amino acid residue 322 as set forth in SEQ ID NO: 1. In some instances, the synthetic molecule is in contact with a residue at an amino acid position corresponding to amino acid residue 257 as set forth in SEQ ID NO: 1. In some instances, the synthetic molecule is in contact with a residue at an amino acid position corresponding to amino acid residue 277 as set forth in SEQ ID NO: 1. In some instances, the synthetic molecule is in contact with a residue at an amino acid position corresponding to amino acid residue 256 as set forth in SEQ ID NO: 1.

In some instances, the modified ENPP polypeptide is an ENPP1 polypeptide. In some instances, the modified ENPP1 polypeptide comprises a synthetic molecule that is in contact with at least one residue at an amino acid position corresponding to amino acid residues 218, 256, 257, 277, 290, 295, 322, 323, 326, 340, 371, 376, 380, 423, 424, or 535 as set forth in SEQ ID NO: 1; in which the synthetic molecule competes with 2-(1-(6,7-Dimethoxyquinazolin-4-yl)piperidin-4-yl)ethyl sulfamide for contact with ENPP1. In some instances, the synthetic molecule is in contact with at least one residue of ENPP1 at an amino acid position corresponding to amino acid residues 218, 256, 257, 277, 322, 326, 340, 376, 380, 423, 424, or 535 as set forth in SEQ ID NO: 1. In some instances, the synthetic molecule is in contact with at least one residue of ENPP1 at an amino acid position corresponding to amino acid residues 256, 257, 277, 322, 323, 326, 340, or 371 as set forth in SEQ ID NO: 1. In some instances, the synthetic molecule is in contact with at least one residue of ENPP1 at an amino acid position corresponding to amino acid residues 256, 257, 277, 322, 323, 340, or 371 as set forth in SEQ ID NO: 1. In some instances, the synthetic molecule is in contact with at least one residue of ENPP1 at an amino acid position corresponding to amino acid residues 257, 322, 326, or 340 as set forth in SEQ ID NO: 1. In some instances, the synthetic molecule is in contact with at least one residue of ENPP1 at an amino acid position corresponding to amino acid residues 257 or 340 as set forth in SEQ ID NO: 1. In some instances, the synthetic molecule is in contact with at least one residue of ENPP1 at an amino acid position corresponding to amino acid residues 326 or 322 as set forth in SEQ ID NO: 1. In some instances, the synthetic molecule is in contact with a residue of ENPP1 at an amino acid position corresponding to amino acid residue 340 as set forth in SEQ ID NO: 1. In some instances, the synthetic molecule is in contact with a residue at an amino acid position corresponding to amino acid residue 323 as set forth in SEQ ID NO: 1. In some instances, the synthetic molecule is in contact with a residue of ENPP1 at an amino acid position corresponding to amino acid residue 322 as set forth in SEQ ID NO: 1. In some instances, the synthetic molecule is in contact with a residue of ENPP1 at an amino acid position corresponding to amino acid residue 257 as set forth in SEQ ID NO: 1. In some instances, the synthetic molecule is in contact with a residue of ENPP1 at an amino acid position corresponding to amino acid residue 277 as set forth in SEQ ID NO: 1. In some instances, the synthetic molecule is in contact with a residue of ENPP1 at an amino acid position corresponding to amino acid residue 256 as set forth in SEQ ID NO: 1. In some instances, the ENPP1 polypeptide is a human ENPP1 polypeptide.

In some instances, the modified ecto-nucleotide pyrophosphatase/phosphodiesterase (ENPP) polypeptide comprising a synthetic molecule that is in contact with at least one residue at an amino acid position corresponding to amino acid residues D218, T256, F257, N277, L290, K295, W322, P323, D326, Y340, Y371, D376, H380, D423, H424, or H535 as set forth in SEQ ID NO: 1; in which the synthetic molecule competes with 2-(1-(6,7-Dimethoxyquinazolin-4-yl)piperidin-4-yl)ethyl sulfamide for contact with ENPP. In some instances, the synthetic molecule is in contact with at least one residue at an amino acid position corresponding to amino acid residues D218, T256, F257, N277, W322, D326, Y340, D376, H380, D423, H424, or H535 as set forth in SEQ ID NO: 1. In some instances, the synthetic molecule is in contact with at least one residue at an amino acid position corresponding to amino acid residues T256, F257, N277, W322, P323, D326, Y340, or Y371 as set forth in SEQ ID NO: 1. In some instances, the synthetic molecule is in contact with at least one residue at an amino acid position corresponding to amino acid residues T256, F257, N277, W322, P323, Y340, or Y371 as set forth in SEQ ID NO: 1. In some instances, the synthetic molecule is in contact with at least one residue at an amino acid position corresponding to amino acid residues F257, W322, D326, or Y340 as set forth in SEQ ID NO: 1. In some instances, the synthetic molecule is in contact with at least one residue at an amino acid position corresponding to amino acid residues F257 or Y340 as set forth in SEQ ID NO: 1. In some instances, the synthetic molecule is in contact with at least one residue at an amino acid position corresponding to amino acid residues D326 or W322 as set forth in SEQ ID NO: 1. In some instances, the synthetic molecule is in contact with a residue at an amino acid position corresponding to amino acid residue Y340 as set forth in SEQ ID NO: 1. In some instances, the synthetic molecule is in contact with a residue at an amino acid position corresponding to amino acid residue P323 as set forth in SEQ ID NO: 1. In some instances, the synthetic molecule is in contact with a residue at an amino acid position corresponding to amino acid residue W322 as set forth in SEQ ID NO: 1. In some instances, the synthetic molecule is in contact with a residue at an amino acid position corresponding to amino acid residue F257 as set forth in SEQ ID NO: 1. In some instances, the synthetic molecule is in contact with a residue at an amino acid position corresponding to amino acid residue N277 as set forth in SEQ ID NO: 1. In some instances, the synthetic molecule is in contact with a residue at an amino acid position corresponding to amino acid residue T256 as set forth in SEQ ID NO: 1.

In some instances, the modified ENPP polypeptide is an ENPP1 polypeptide. In some instances, the modified ENPP1 polypeptide comprises a synthetic molecule that is in contact with at least one residue at an amino acid position corresponding to amino acid residues D218, T256, F257, N277, L290, K295, W322, P323, D326, Y340, Y371, D376, H380, D423, H424, or H535 as set forth in SEQ ID NO: 1; in which the synthetic molecule competes with 2-(1-(6,7-Dimethoxyquinazolin-4-yl)piperidin-4-yl)ethyl sulfamide for contact with ENPP1. In some instances, the synthetic molecule is in contact with at least one residue of ENPP1 at an amino acid position corresponding to amino acid residues D218, T256, F257, N277, W322, D326, Y340, D376, H380, D423, H424, or H535 as set forth in SEQ ID NO: 1. In some instances, the synthetic molecule is in contact with at least one residue of ENPP1 at an amino acid position corresponding to amino acid residues T256, F257, N277, W322, P323, D326, Y340, or Y371 as set forth in SEQ ID NO: 1. In some instances, the synthetic molecule is in contact with at least one residue of ENPP1 at an amino acid position corresponding to amino acid residues T256, F257, N277, W322, P323, Y340, or Y371 as set forth in SEQ ID NO: 1. In some instances, the synthetic molecule is in contact with at least one residue of ENPP1 at an amino acid position corresponding to amino acid residues F257, W322, D326, or Y340 as set forth in SEQ ID NO: 1. In some instances, the synthetic molecule is in contact with at least one residue of ENPP1 at an amino acid position corresponding to amino acid residues F257 or Y340 as set forth in SEQ ID NO: 1. In some instances, the synthetic molecule is in contact with at least one residue of ENPP1 at an amino acid position corresponding to amino acid residues D326 or W322 as set forth in SEQ ID NO: 1. In some instances, the synthetic molecule is in contact with a residue of ENPP1 at an amino acid position corresponding to amino acid residue Y340 as set forth in SEQ ID NO: 1. In some instances, the synthetic molecule is in contact with a residue of ENPP1 at an amino acid position corresponding to amino acid residue P323 as set forth in SEQ ID NO: 1. In some instances, the synthetic molecule is in contact with a residue of ENPP1 at an amino acid position corresponding to amino acid residue W322 as set forth in SEQ ID NO: 1. In some instances, the synthetic molecule is in contact with a residue of ENPP1 at an amino acid position corresponding to amino acid residue F257 as set forth in SEQ ID NO: 1. In some instances, the synthetic molecule is in contact with a residue of ENPP1 at an amino acid position corresponding to amino acid residue N277 as set forth in SEQ ID NO: 1. In some instances, the synthetic molecule is in contact with a residue of ENPP1 at an amino acid position corresponding to amino acid residue T256 as set forth in SEQ ID NO: 1. In some instances, the ENPP1 polypeptide is a human ENPP1 polypeptide.

In some embodiments, the contact between an amino acid residue and the synthetic molecule comprises a covalent interaction or a non-covalent interaction. In some instances, the contact between an amino acid residue and the synthetic molecule comprises a hydrogen bonding, a hydrophobic interaction, an ionic interaction, a Van der Waals interaction, an electrostatic interaction, or a pi-pi bonding.

In some embodiments, the synthetic molecule is not a hydrolysis product of a nucleoside triphosphate. In some instances, the nucleoside triphosphate comprises adenosine triphosphate (ATP), thymidine triphosphate (TTP), guanosine triphosphate (GTP), or cytidine triphosphate (CTP). In some instances, the hydrolysis product is adenosine monophosphate (AMP), thymidine monophosphate (TMP), guanosine monophosphate (GMP), or cytidine monophosphate (CMP).

### Synthetic Molecules

Described herein are synthetic compounds of Formula (I′), (I), (II), (III), (IV), or (V) that are ENPP inhibitors (e.g., ENPP-1 inhibitors).

Disclosed herein is a compound of Formula (I′), or a pharmaceutically acceptable salt, solvate, or stereoisomer thereof:

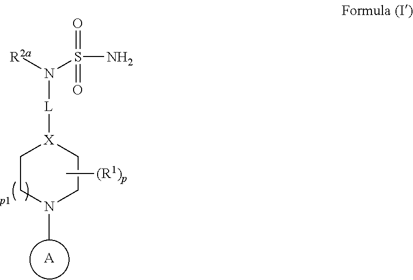

wherein
L is —(CR³R⁴)ₙ—;
X is —N— or —CH—;
Ring A is
   (a) an optionally substituted aryl or cycloalkyl;
   (b) an optionally substituted heteroaryl that is not quinazolinyl or pyrimidyl; or
   (c) an optionally substituted heterocycloalkyl; or
   (d) a ring selected from

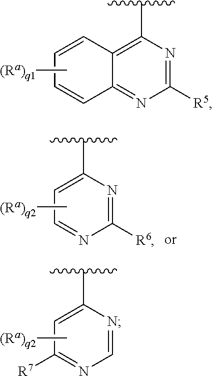

each R¹ is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, and optionally substituted (C₁-C₆ alkyl)heteroaryl;
or two R¹ on the same carbon are taken together to form an oxo;
R^{2a} is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R³ and R⁴ are independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R³ and R⁴ on the same carbon are taken together to form an oxo;
R⁵ is halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ heteroalkyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R⁶ is hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl; provided that R⁶ is not substituted imidazolyl;
R⁷ is hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted (C₁-C₆ alkyl)aryl, optionally substituted heteroaryl, or optionally substituted (C₁-C₆ alkyl)heteroaryl; provided that R⁷ is not substituted imidazolyl;
each R^{a} is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
n is 1-4;
p is 1-4;
p1 is 0 or 1;
q1 is 1-4; and
q2 is 1-2.

Disclosed herein is a compound of Formula (I), or a pharmaceutically acceptable salt, solvate, or stereoisomer thereof:

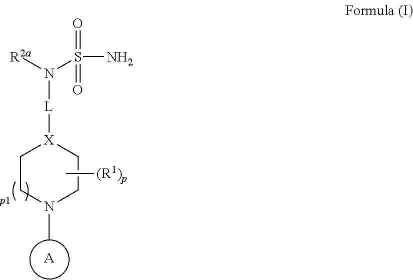

wherein
L is —(CR³R⁴)ₙ—;
X is —N— or —CH—;
Ring A is
   (a) an optionally substituted heteroaryl that is not quinazolinyl or pyrimidyl; or
   (b) an optionally substituted heterocycloalkyl; or
   (c) a ring selected from

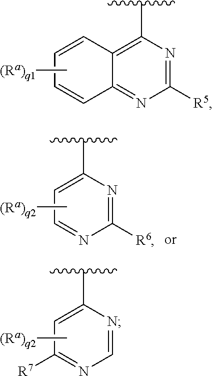

each R¹ is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, and optionally substituted (C₁-C₆ alkyl)heteroaryl;
or two R¹ on the same carbon are taken together to form an oxo;
R^{2a} is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R³ and R⁴ are independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R³ and R⁴ on the same carbon are taken together to form an oxo;
R⁵ is halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ heteroalkyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R⁶ is hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl; provided that R⁶ is not substituted imidazolyl;
R⁷ is hydrogen, halogen, —CN, —OR¹¹, —SR¹¹, —S(═O)R₁₀, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted (C₁-C₆ alkyl)aryl, optionally substituted heteroaryl, or optionally substituted (C₁-C₆ alkyl)heteroaryl; provided that R⁷ is not substituted imidazolyl;
each R^{a} is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
n is 1-4;
p is 1-4;
p1 is 0 or 1;
q1 is 1-4; and
q2 is 1-2.

In some embodiments of a compound of Formula (I′) or (I), R^{2a} is hydrogen or optionally substituted C₁-C₆ alkyl. In some embodiments of a compound of Formula (I′) or (I), R^{2a} is hydrogen, C₁-C₆ alkyl, or C₁-C₆ haloalkyl. In some embodiments of a compound of Formula (I′) or (I), R^{2a} is hydrogen.

In some embodiments of a compound of Formula (I′) or (I), n is 1 or 2. In some embodiments of a compound of Formula (I′) or (I), n is 1. In some embodiments of a compound of Formula (I′) or (I), n is 2. In some embodiments of a compound of Formula (I′) or (I), n is 3. In some embodiments of a compound of Formula (I′) or (I), n is 4.

In some embodiments of a compound of Formula (I′) or (I), each R³ and R⁴ are independently hydrogen, halogen, —CN, —OR¹¹, —NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (I′) or (I), each R³ and R⁴ are independently hydrogen, halogen, —CN, —OH, or optionally substituted C₁-C₆ alkyl. In some embodiments of a compound of Formula (I′) or (I), each R³ and R⁴ are independently hydrogen, halogen, C₁-C₆ alkyl, or C₁-C₆ haloalkyl. In some embodiments of a compound of Formula (I′) or (I), each R³ and R⁴ are independently hydrogen or halogen. In some embodiments of a compound of Formula (I′) or (I), each R³ and R⁴ are hydrogen. In some embodiments of a compound of Formula (I′) or (I), R³ and R⁴ on the same carbon are taken together to form an oxo.

In some embodiments of a compound of Formula (I′) or (I), L is —(CR³R⁴)ₙ—; n is 2; and each R³ and R⁴ are independently hydrogen or halogen.

In some embodiments of a compound of Formula (I′) or (I), X is —CH—. In some embodiments of a compound of Formula (I′) or (I), X is —N—.

In some embodiments of a compound of Formula (I′) or (I), p1 is 1. In some embodiments of a compound of Formula (I′) or (I), p is 0.

In some embodiments of a compound of Formula (I′) or (I), p is 1 or 2. In some embodiments of a compound of Formula (I′) or (I), p is 1. In some embodiments of a compound of Formula (I′) or (I), p is 2. In some embodiments of a compound of Formula (I′) or (I), p is 3. In some embodiments of a compound of Formula (I′) or (I), p is 4.

In some embodiments of a compound of Formula (I′) or (I), each R¹ is independently hydrogen, halogen, —CN, —OR¹¹, —NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (I′) or (I), each R¹ is independently hydrogen, halogen, —CN, —OH, or optionally substituted C₁-C₆ alkyl. In some embodiments of a compound of Formula (I′) or (I), each R¹ is independently hydrogen, halogen, or optionally substituted C₁-C₆ alkyl. In some embodiments of a compound of Formula (I′) or (I), each R¹ is independently hydrogen, halogen, C₁-C₆ alkyl, or C₁-C₆ haloalkyl. In some embodiments of a compound of Formula (I′) or (I), each R¹ is hydrogen.

In some embodiments of a compound of Formula (I′), Ring A is aryl. In some embodiments of a compound of Formula (I′), Ring A is cycloalkyl.

In some embodiments of a compound of Formula (I′) or (I), Ring A is selected from:

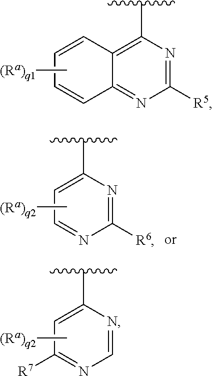

optionally substituted pyridinyl, optionally substituted pyrazinyl, optionally substituted pyridazinyl, optionally substituted pyrrolyl, optionally substituted pyrazolyl, optionally substituted imidazolyl, optionally substituted triazolyl, optionally substituted tetrazolyl, optionally substituted isoxazolyl, optionally substituted oxazolyl, optionally substituted isothiazolyl, optionally substituted thiazolyl, optionally substituted quinolinyl, optionally substituted isoquinolinyl, optionally substituted naphthyridinyl, optionally substituted cinnolinyl, optionally substituted pyridopyridazinyl, optionally substituted phthalazinyl, optionally substituted indolyl, optionally substituted pyrrolopyridinyl, optionally substituted indazolyl, optionally substituted pyrazolopyridine, optionally substituted benzotriazolyl, optionally substituted benzimidazolyl, optionally substituted pyrrolopyrimidinyl, optionally substituted pyrazolopyrimidinyl, optionally substituted triazolopyrimidinyl, optionally substituted purinyl, optionally substituted pyrrolopyridinyl, optionally substituted pyrazolopyridinyl, optionally substituted triazolopyridinyl, optionally substituted imidazopyridinyl, optionally substituted pyrrolo[2,1-f][1,2,4]triazinyl, optionally substituted pyrazolo[5,1-f][1,2,4]triazinyl, optionally substituted imidazo[5,1-f][1,2,4]triazinyl, optionally substituted imidazo[2,1-f][1,2,4]triazinyl, optionally substituted pyrrolo[1,2-a]pyrazinyl, optionally substituted pyrazolo[1,5-a]pyrazinyl, optionally substituted imidazo[1,5-a]pyrazinyl, optionally substituted imidazo[1,2-a]pyrazinyl, optionally substituted pyrrolo[1,2-c]pyrimidinyl, optionally substituted pyrazolo[1,5-c]pyrimidinyl, optionally substituted imidazo[1,5-c]pyrimidinyl, optionally substituted imidazo[1,2-c]pyrimidinyl, optionally substituted pyrrolo[1,2-b]pyridazinyl, optionally substituted pyrazolo[1,5-b]pyridazinyl, optionally substituted imidazo[1,5-b]pyridazinyl, optionally substituted imidazo[1,2-b]pyridazinyl, optionally substituted indolizinyl, optionally substituted pyrazolo[1,5-a]pyridinyl, optionally substituted imidazo[1,5-a]pyridinyl, optionally substituted imidazo[1,5-a]pyridinyl, optionally substituted imidazo[1,2-a]pyridinyl, optionally substituted pyrrolo[1,2-a][1,3,5]triazinyl, optionally substituted pyrazolo[1,5-a][1,3,5]triazinyl, optionally substituted imidazo[1,5-a][1,3,5]triazinyl, optionally substituted imidazo[1,2-a][1,3,5]triazinyl, optionally substituted pyrrolo[1,2-c]pyrimidinyl, optionally substituted pyrazolo[1,5-c]pyrimidinyl, optionally substituted imidazo[1,5-c]pyrimidinyl, optionally substituted imidazo[1,2-c]pyrimidinyl, optionally substituted pyrrolo[1,2-a]pyrazinyl, optionally substituted pyrazolo[1,5-a]pyrazinyl, optionally substituted imidazo[1,5-a]pyrazinyl, optionally substituted imidazo[1,2-a]pyrazinyl, optionally substituted pyrrolo[1,2-a]pyrimidinyl, optionally substituted pyrazolo[1,5-a]pyrimidinyl, optionally substituted imidazo[1,5-a]pyrimidinyl, optionally substituted imidazo[1,2-a]pyrimidinyl, optionally substituted tetrahydroquinazolinyl, optionally substituted dihydropyranopyrimidinyl, optionally substituted tetrahydropyridopyrimidinyl, optionally substituted tetrahydroquinolinyl, optionally substituted dihydropyranopyridinyl, optionally substituted tetrahydronaphthyridinyl, optionally substituted tetrahydroisoquinolinyl, optionally substituted dihydropyranopyridinyl, optionally substituted tetrahydronaphthyridinyl, optionally substituted dihydropurinone, optionally substituted dihydroimidazopyridinone, optionally substituted dihydrobenzoimidazolone, optionally substituted dihydropyrrolopyrimidinone, optionally substituted dihydropyrrolopyridinone, and optionally substituted indolinone.

In some embodiments of a compound of Formula (I′) or (I), Ring A is selected from:

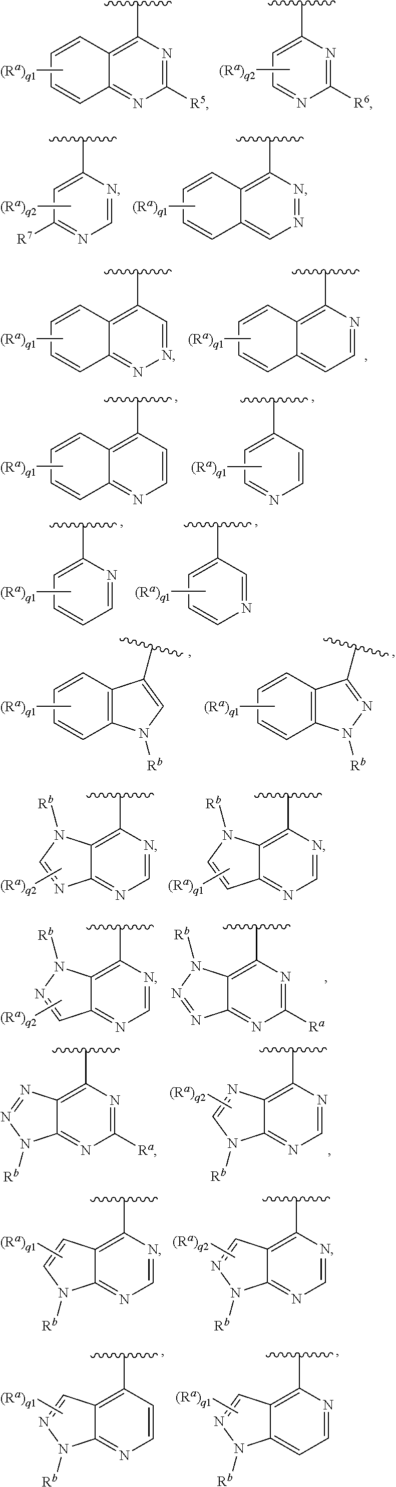

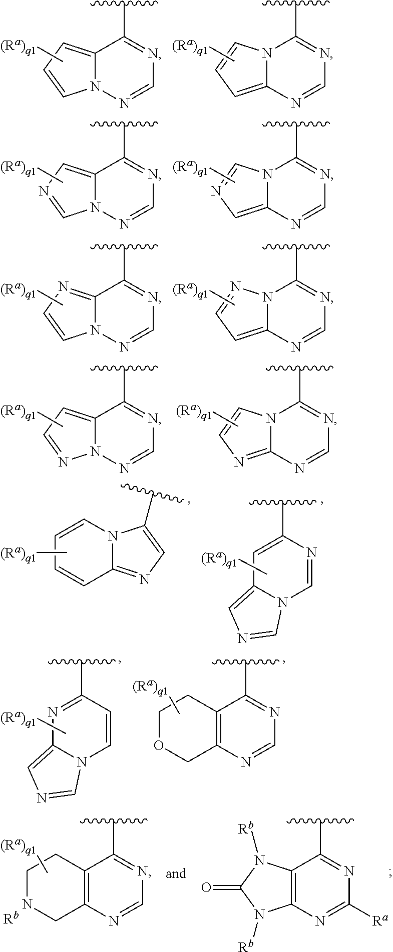

and

each R^{b} is independently hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl.

In some embodiments of a compound of Formula (I′) or (I), Ring A is selected from:

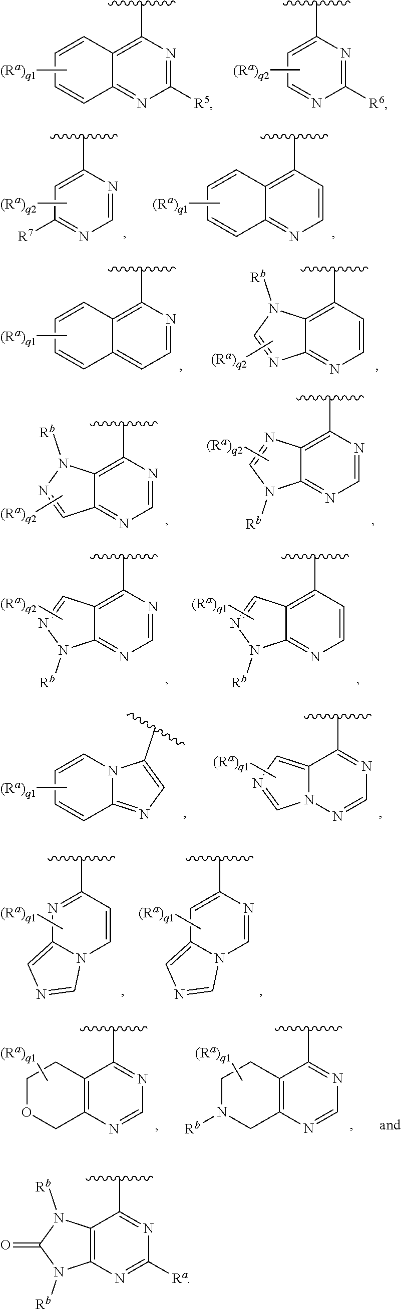

In some embodiments of a compound of Formula (I′) or (I), Ring A is

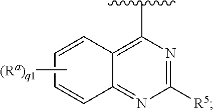

each R^{a} is independently hydrogen, halogen, —CN, —OR¹¹, optionally substituted C₁-C₆ alkyl, or optionally substituted C₁-C₆ heteroalkyl; and q1 is 2 or 3. In some embodiments of a compound of Formula (I′) or (I), Ring A is

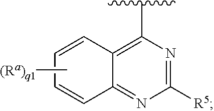

each R^{a} is —OR¹¹; and q1 is 2.

In some embodiments of a compound of Formula (I′) or (I), Ring A is

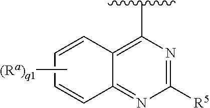

and R⁵ is halogen, —CN, —OR¹¹, —NR¹¹R¹², —C(═O)OR¹¹, —NR¹¹C(═O)R¹⁰, optionally substituted C₁-C₆ heteroalkyl, optionally substituted cycloalkyl, optionally substituted heterocycloalkyl, optionally substituted aryl, or optionally substituted heteroaryl. In some embodiments of a compound of Formula (I′) or (I), Ring A is

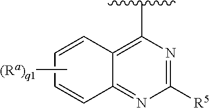

and R⁵ is —NR¹¹R¹², —NR¹¹C(═O)R¹⁰, optionally substituted aryl, or optionally substituted heteroaryl.

In some embodiments of a compound of Formula (I′) or (I), Ring A is

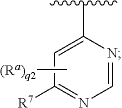

each R^{a} is independently hydrogen, halogen, —CN, —OR¹¹, optionally substituted C₁-C₆ alkyl, or optionally substituted C₁-C₆ heteroalkyl; and q2 is 1. In some embodiments of a compound of Formula (I′) or (I), Ring A is

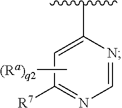

R^{a} is hydrogen or C₁-C₆ alkyl; and q2 is 1.

In some embodiments of a compound of Formula (I′) or (I), Ring A is

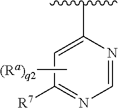

and R⁷ is hydrogen, halogen, —CN, —OR¹¹, —NR¹¹R¹², —NR¹¹C(═O)R¹⁰, —C(═O)NR¹¹R¹², optionally substituted C₁-C₆ alkyl, optionally substituted C₁-C₆ heteroalkyl, optionally substituted cycloalkyl, optionally substituted heterocycloalkyl, optionally substituted aryl, or optionally substituted heteroaryl; provided that R⁷ is not substituted imidazolyl. In some embodiments of a compound of Formula (I′) or (I), Ring A is

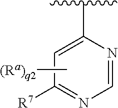

and R⁷ is optionally substituted C₁-C₆ alkyl, optionally substituted aryl, or optionally substituted heteroaryl; provided that R⁷ is not substituted imidazolyl. In some embodiments of a compound of Formula (I′) or (I), Ring A is

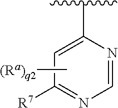

and R⁷ is optionally substituted C₁-C₆ alkyl or optionally substituted aryl.

In some embodiments of a compound of Formula (I′) or (I), Ring A is

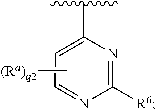

each R^{a} is independently hydrogen, halogen, —CN, —OR¹¹, optionally substituted C₁-C₆ alkyl, or optionally substituted C₁-C₆ heteroalkyl; and q2 is 1. In some embodiments of a compound of Formula (I′) or (I), Ring A is

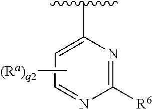

and each R^{a} is hydrogen.

In some embodiments of a compound of Formula (I′) or (I), Ring A is

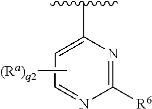

and R⁶ is hydrogen, halogen, —CN, —OR¹¹, —NR¹¹R¹², —NR¹¹C(═O)R¹⁰, optionally substituted C₁-C₆ alkyl, optionally substituted C₁-C₆ heteroalkyl, optionally substituted cycloalkyl, optionally substituted heterocycloalkyl, optionally substituted aryl, or optionally substituted heteroaryl; provided that R⁶ is not substituted imidazolyl. In some embodiments of a compound of Formula (I′) or (I), Ring A is

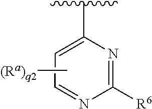

and R⁶ is hydrogen, —NR¹¹R¹², —NR¹¹C(═O)R¹⁰, or optionally substituted heteroaryl; provided that R⁶ is not substituted imidazolyl. In some embodiments of a compound of Formula (I′) or (I), Ring A is

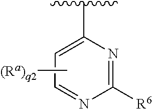

and R⁶ is hydrogen, —NR¹¹R¹², or —NR¹¹C(═O)R¹⁰.

In some embodiments of a compound of Formula (I′) or (I), Ring A is selected from:

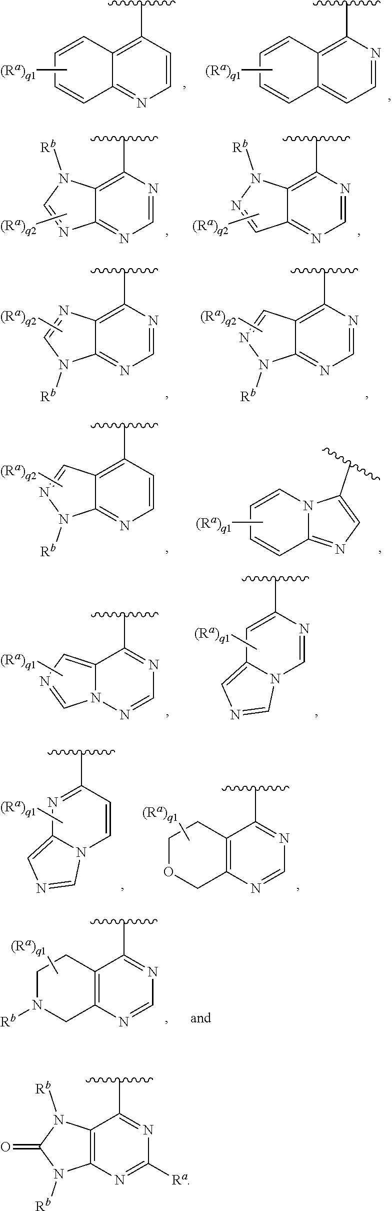

In some embodiments of a compound of Formula (I′) or (I), q1 is 1 or 2. In some embodiments of a compound of Formula (I′) or (I), q1 is 1-3. In some embodiments of a compound of Formula (I′) or (I), q1 is 1. In some embodiments of a compound of Formula (I′) or (I), q1 is 2. In some embodiments of a compound of Formula (I′) or (I), q1 is 3. In some embodiments of a compound of Formula (I′) or (I), q1 is 4. In some embodiments of a compound of Formula (I′) or (I), q2 is 1 or 2. In some embodiments of a compound of Formula (I′) or (I), q2 is 1. In some embodiments of a compound of Formula (I′) or (I), q2 is 2.

In some embodiments of a compound of Formula (I′) or (I), each R^{a} is independently hydrogen, halogen, —CN, —OR¹¹, —NR¹¹R¹², —C(═O)OR¹¹, —OC(═O)OR¹¹, —C(═O)NR¹¹R¹², optionally substituted C₁-C₆ alkyl, optionally substituted C₁-C₆ heteroalkyl, optionally substituted cycloalkyl, or optionally substituted heterocycloalkyl.

In some embodiments of a compound of Formula (I′) or (I), each R^{b} is independently hydrogen, optionally substituted C₁-C₆ alkyl, or optionally substituted aryl.

Also disclosed herein is a compound of Formula (II), or a pharmaceutically acceptable salt or solvate thereof:

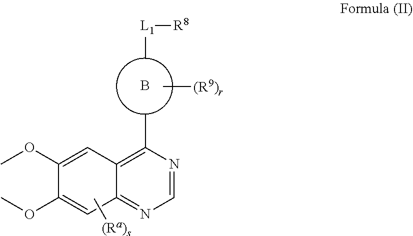

wherein
L₁ is a bond or —(CR¹³R¹⁴)ₙ₁—;
R⁸ is —S(═O)₂NH₂ or —NR^{2b}S(═O)₂NH₂;
Ring B is a bicyclic ring or a 5-membered heteroaryl ring; provided that Ring B is not triazolyl when R⁸ is —NHS(═O)₂NH₂;
R^{2b} is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R⁹ is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
or two R⁹ on the same carbon are taken together to form an oxo;
each R¹³ and R¹⁴ are independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R¹³ and R¹⁴ on the same carbon are taken together to form an oxo;
each R^{a} is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
n1 is 1 or 2;
r is 1-4; and
s is 1-3.

In some embodiments of a compound of Formula (II), s is 1 or 2. In some embodiments of a compound of Formula (II), s is 1. In some embodiments of a compound of Formula (II), s is 2. In some embodiments of a compound of Formula (II), s is 3.

In some embodiments of a compound of Formula (II), each R^{a} is independently hydrogen, halogen, —CN, —OR¹¹, —NR¹¹R¹², —C(═O)OR¹¹, —OC(═O)OR¹¹, —C(═O)NR¹¹R¹², optionally substituted C₁-C₆ alkyl, optionally substituted C₁-C₆ heteroalkyl, optionally substituted cycloalkyl, or optionally substituted heterocycloalkyl. In some embodiments of a compound of Formula (II), each R^{a} is independently hydrogen, halogen, —CN, —OH, optionally substituted C₁-C₆ alkyl, or optionally substituted C₁-C₆ heteroalkyl. In some embodiments of a compound of Formula (II), each R^{a} is independently hydrogen, halogen, or optionally substituted C₁-C₆ alkyl. In some embodiments of a compound of Formula (II), each R^{a} is independently hydrogen, halogen, C₁-C₆ alkyl, or C₁-C₆ haloalkyl.

In some embodiments of a compound of Formula (II), each R^{a} is independently hydrogen, halogen, —CN, —OR¹¹, —NR¹¹R¹², —C(═O)OR¹¹, —OC(═O)OR¹¹, —C(═O)NR¹¹R¹², optionally substituted C₁-C₆ alkyl, optionally substituted C₁-C₆ heteroalkyl, optionally substituted cycloalkyl, or optionally substituted heterocycloalkyl; and s is 1 or 2.

In some embodiments of a compound of Formula (II), each R^{a} is hydrogen.

In some embodiments of a compound of Formula (II), n1 is 1. In some embodiments of a compound of Formula (II), n1 is 2.

In some embodiments of a compound of Formula (II), each R¹³ and R¹⁴ are independently hydrogen, halogen, —CN, —OR¹¹, —NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (II), each R¹³ and R¹⁴ are independently hydrogen, halogen, —CN, —OH, or optionally substituted C₁-C₆ alkyl. In some embodiments of a compound of Formula (II), each R¹³ and R¹⁴ are independently hydrogen, halogen, C₁-C₆ alkyl, or C₁-C₆ haloalkyl. In some embodiments of a compound of Formula (II), each R¹³ and R¹⁴ are independently hydrogen or halogen. In some embodiments of a compound of Formula (II), each R¹³ and R¹⁴ are hydrogen. In some embodiments of a compound of Formula (II), R¹³ and R¹⁴ on the same carbon are taken together to form an oxo.

In some embodiments of a compound of Formula (II), L₁ is —(CR¹³R¹⁴)ₙ₁—; n1 is 1; and each R¹³ and R¹⁴ are independently hydrogen or halogen.

In some embodiments of a compound of Formula (II), L₁ is a bond.

In some embodiments of a compound of Formula (II), Ring B is a fused bicyclic ring. In some embodiments of a compound of Formula (II), Ring B is a spiro bicyclic ring. In some embodiments of a compound of Formula (II), Ring B is selected from

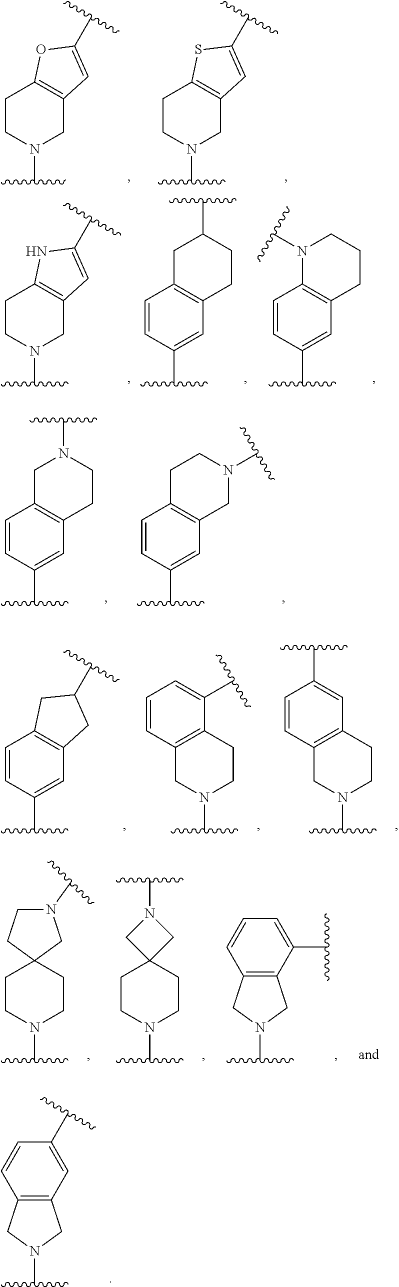

In some embodiments of a compound of Formula (II), Ring B is a 5-membered heteroaryl selected from thiophenyl, furanyl, pyrrolyl, thiazolyl, oxazolyl, imidazolyl, pyrazolyl, isoxazolyl, and isothiazolyl.

In some embodiments of a compound of Formula (II), r is 1 or 2. In some embodiments of a compound of Formula (II), r is 1. In some embodiments of a compound of Formula (II), r is 2. In some embodiments of a compound of Formula (II), r is 3. In some embodiments of a compound of Formula (II), r is 4.

In some embodiments of a compound of Formula (II), each R⁹ is independently hydrogen, halogen, —CN, —OR¹¹, —NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (II), each R⁹ is independently hydrogen, halogen, —CN, —OH, or optionally substituted C₁-C₆ alkyl. In some embodiments of a compound of Formula (II), each R⁹ is independently hydrogen, halogen, or optionally substituted C₁-C₆ alkyl. In some embodiments of a compound of Formula (II), each R⁹ is independently hydrogen, halogen, C₁-C₆ alkyl, or C₁-C₆ haloalkyl. In some embodiments of a compound of Formula (II), each R⁹ is hydrogen.

In some embodiments of a compound of Formula (II), R⁸ is —S(═O)₂NH₂.

In some embodiments of a compound of Formula (II), R^{2b} is hydrogen or optionally substituted C₁-C₆ alkyl. In some embodiments of a compound of Formula (II), R^{2b} is hydrogen, C₁-C₆ alkyl, or C₁-C₆ haloalkyl. In some embodiments of a compound of Formula (II), R^{2b} is hydrogen. In some embodiments of a compound of Formula (II), R⁸ is —NR^{2b}S(═O)₂NH₂; and R^{2b} is hydrogen.

Also disclosed herein is a compound of Formula (III), or a pharmaceutically acceptable salt or solvate thereof:

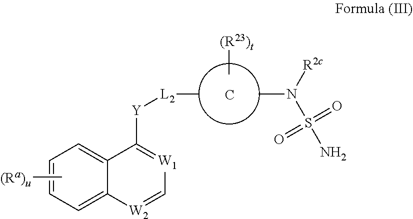

wherein
Y is —O— or —NR²⁰—;
L₂ is a bond or —(CR²¹R²²)ₙ₂—;
W₁ and W₂ are independently N or CR^{a}; provided that at least one of W₁ or W₂ is N;
Ring C is aryl, heteroaryl, cycloalkyl, or heterocycloalkyl;
each R²³ is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R^{2c} is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R²⁰ is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R²¹ and R²² are independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R²¹ and R²² on the same carbon are taken together to form an oxo;
each R^{a} is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
t is 1-4;
n2 is 1 or 2; and
u is 1-4.

In some embodiments of a compound of Formula (III), W₁ and W₂ are N.

In some embodiments of a compound of Formula (III), W₁ is N; and W₂ is CR^{a}.

In some embodiments of a compound of Formula (III), W₁ is CR^{a}; and W₂ is N.

In some embodiments of a compound of Formula (III), u is 1-3. In some embodiments of a compound of Formula (III), u is 1 or 2. In some embodiments of a compound of Formula (III), u is 1. In some embodiments of a compound of Formula (III), u is 2. In some embodiments of a compound of Formula (III), u is 3. In some embodiments of a compound of Formula (III), u is 4.

In some embodiments of a compound of Formula (III), each R^{a} is independently hydrogen, halogen, —CN, —OR¹¹, —NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (III), each R^{a} is independently hydrogen, halogen, —CN, —OR¹¹, —NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (III), each R^{a} is independently hydrogen, halogen, —OR¹¹, or optionally substituted C₁-C₆ alkyl. In some embodiments of a compound of Formula (III), each R^{a} is independently hydrogen, halogen, —OR¹¹, C₁-C₆ alkyl, or C₁-C₆ haloalkyl.

In some embodiments of a compound of Formula (III), each R^{a} is independently hydrogen, halogen, —CN, —OR¹¹, —NR¹¹R¹², —C(═O)OR¹¹, —OC(═O)OR¹¹, —C(═O)NR¹¹R¹², optionally substituted C₁-C₆ alkyl, optionally substituted C₁-C₆ heteroalkyl, optionally substituted cycloalkyl, or optionally substituted heterocycloalkyl; and u is 1-3. In some embodiments of a compound of Formula (III), each R^{a} is —OR¹¹; and u is 1 or 2.

In some embodiments of a compound of Formula (III), t is 1 or 2. In some embodiments of a compound of Formula (III), t is 1. In some embodiments of a compound of Formula (III), t is 2. In some embodiments of a compound of Formula (III), t is 3. In some embodiments of a compound of Formula (III), t is 4.

In some embodiments of a compound of Formula (III), each R²³ is independently hydrogen, halogen, —CN, —OR¹¹, —NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (III), each R²³ is independently hydrogen, halogen, —CN, —OH, or optionally substituted C₁-C₆ alkyl. In some embodiments of a compound of Formula (III), each R²³ is independently hydrogen, halogen, or optionally substituted C₁-C₆ alkyl. In some embodiments of a compound of Formula (III), each R²³ is independently hydrogen, halogen, C₁-C₆ alkyl, or C₁-C₆ haloalkyl. In some embodiments of a compound of Formula (III), each R²³ is hydrogen.

In some embodiments of a compound of Formula (III), Y is —NR²⁰—.

In some embodiments of a compound of Formula (III), R²⁰ is hydrogen or optionally substituted C₁-C₆ alkyl. In some embodiments of a compound of Formula (III), R²⁰ is hydrogen, C₁-C₆ alkyl, or C₁-C₆ haloalkyl. In some embodiments of a compound of Formula (III), R²⁰ is hydrogen or C₁-C₆ alkyl.

In some embodiments of a compound of Formula (III), Y is —O—.

In some embodiments of a compound of Formula (III), L₂ is a bond.

In some embodiments of a compound of Formula (III), n2 is 1. In some embodiments of a compound of Formula (III), n2 is 2.

In some embodiments of a compound of Formula (III), each R² and R²² are independently hydrogen, halogen, —CN, —OR¹¹, —NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (III), each R²¹ and R²² are independently hydrogen, halogen, —CN, —OH, or optionally substituted C₁-C₆ alkyl. In some embodiments of a compound of Formula (III), each R² and R²² are independently hydrogen, halogen, C₁-C₆ alkyl, or C₁-C₆ haloalkyl. In some embodiments of a compound of Formula (III), each R² and R²² are independently hydrogen or halogen. In some embodiments of a compound of Formula (III), each R²¹ and R²² are hydrogen. In some embodiments of a compound of Formula (III), R²¹ and R²² on the same carbon are taken together to form an oxo.

In some embodiments of a compound of Formula (III), L₂ is —(CR²¹R²²)ₙ₂—; n2 is 1 or 2; and each R²¹ and R²² are independently hydrogen or halogen.

In some embodiments of a compound of Formula (III), R^{2c} is hydrogen or optionally substituted C₁-C₆ alkyl. In some embodiments of a compound of Formula (III), R^{2c} is hydrogen, C₁-C₆ alkyl, or C₁-C₆ haloalkyl. In some embodiments of a compound of Formula (III), R^{2c} is hydrogen or C₁-C₆ alkyl. In some embodiments of a compound of Formula (III), R^{2c} is hydrogen.

In some embodiments of a compound of Formula (III), Ring C is an aryl. In some embodiments of a compound of Formula (III), Ring C is a 6-membered aryl. In some embodiments of a compound of Formula (III), Ring C is phenyl.

In some embodiments of a compound of Formula (III), Ring C is a heteroaryl. In some embodiments of a compound of Formula (III), Ring C is a 5-membered heteroaryl. In some embodiments of a compound of Formula (III), Ring C is a 5-membered heteroaryl selected from thiophenyl, furanyl, pyrrolyl, thiazolyl, oxazolyl, imidazolyl, pyrazolyl, isoxazolyl, and isothiazolyl. In some embodiments of a compound of Formula (III), Ring C is a 5-membered heteroaryl selected from thiophenyl, furanyl, thiazolyl, and oxazolyl. In some embodiments of a compound of Formula (III), Ring C is a 6-membered heteroaryl. In some embodiments of a compound of Formula (III), Ring C is pyridinyl or pyrimidyl.

In some embodiments of a compound of Formula (III), Ring C is a cycloalkyl. In some embodiments of a compound of Formula (III), Ring C is a cycloalkyl selected from cyclopropyl, cyclobutyl, cyclopentyl, and cyclohexyl.

In some embodiments of a compound of Formula (III), Ring C is a heterocycloalkyl. In some embodiments of a compound of Formula (III), Ring C is a heterocycloalkyl selected from pyrrolidinyl, piperidinyl, piperazinyl, or morpholinyl.

Disclosed herein is a compound of Formula (IV), or a pharmaceutically acceptable salt, solvate, or stereoisomer thereof:

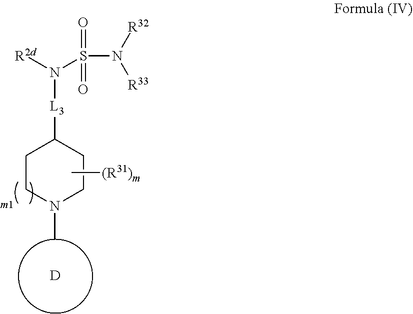

wherein
L₃ is —(CR³⁴R³⁵)ₙ₃—;
Ring D is optionally substituted heteroaryl or optionally substituted heterocycloalkyl;
each R³¹ is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, and optionally substituted (C₁-C₆ alkyl)heteroaryl;
or two R³¹ on the same carbon are taken together to form an oxo;
R^{2d} is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R³² and R³³ are independently optionally substituted C₁-C₆ alkyl;
or R³² and R³³ taken together form an optionally substituted heterocycloalkyl;
each R³⁴ and R³⁵ are independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R³⁴ and R³⁵ on the same carbon are taken together to form an oxo;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
n3 is 1-4;
m is 1-4; and
m1 is 0 or 1.

In some embodiments of a compound of Formula (IV), Ring D is optionally substituted heteroaryl. In some embodiments of a compound of Formula (IV), Ring D is optionally substituted heteroaryl selected from quinolinyl, isoquinolinyl, quinazolinyl, naphthyridinyl, cinnolinyl, pyridopyridazinyl, phthalazinyl, indolyl, pyrrolopyridinyl, indazolyl, pyrazolopyridine, benzotriazolyl, benzimidazolyl, pyrrolopyrimidinyl, pyrazolopyrimidinyl, triazolopyrimidinyl, purinyl, pyrrolopyridinyl, pyrazolopyridinyl, triazolopyridinyl, and imidazopyridinyl. In some embodiments of a compound of Formula (IV), Ring D is optionally substituted heteroaryl selected from 2-pyridinyl, 3-pyridinyl, 4-pyridimidyl, 5-pyridimidyl, and 2-pyrazinyl. In some embodiments of a compound of Formula (IV), Ring D is heteroaryl optionally substituted with one, two, or three halogen, —CN, —OR¹¹, —NR¹¹R¹², —C(═O)OR¹¹, —C(═O)NR¹¹R¹², optionally substituted C₁-C₆ alkyl, optionally substituted C₁-C₆ heteroalkyl, optionally substituted cycloalkyl, optionally substituted heterocycloalkyl, optionally substituted aryl, or optionally substituted heteroaryl.

In some embodiments of a compound of Formula (IV), Ring D is optionally substituted heterocycloalkyl. In some embodiments of a compound of Formula (IV), Ring D is optionally substituted heterocycloalkyl selected from pyrrolidinyl, piperidinyl, piperazinyl, and morpholinyl. In some embodiments of a compound of Formula (IV), Ring D is optionally substituted heterocycloalkyl selected from pyrrolidinyl, piperazinyl, and morpholinyl.

In some embodiments of a compound of Formula (IV), R³² and R³³ are independently optionally substituted C₁-C₆ alkyl.

In some embodiments of a compound of Formula (IV), R³² and R³³ taken together form an optionally substituted heterocycloalkyl. In some embodiments of a compound of Formula (IV), R³² and R³³ taken together form an optionally substituted heterocycloalkyl selected from pyrrolidinyl, piperidinyl, piperazinyl, and morpholinyl.

In some embodiments of a compound of Formula (IV), each R³⁴ and R³⁵ are independently hydrogen, halogen, —CN, —OR¹¹, —NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (IV), each R³⁴ and R³⁵ are independently hydrogen, halogen, —CN, —OH, or optionally substituted C₁-C₆ alkyl. In some embodiments of a compound of Formula (IV), each R³⁴ and R³⁵ are independently hydrogen, halogen, C₁-C₆ alkyl, or C₁-C₆ haloalkyl. In some embodiments of a compound of Formula (IV), each R³⁴ and R³⁵ are independently hydrogen or halogen. In some embodiments of a compound of Formula (IV), each R³⁴ and R³⁵ are hydrogen. In some embodiments of a compound of Formula (IV), R³⁴ and R³⁵ on the same carbon are taken together to form an oxo.

In some embodiments of a compound of Formula (IV), L₃ is —(CR³⁴R³⁵)ₙ₃—; n3 is 1 or 2; and each R³⁴ and R³⁵ are independently hydrogen or halogen.

In some embodiments of a compound of Formula (IV), m1 is 0. In some embodiments of a compound of Formula (IV), m1 is 1.

In some embodiments of a compound of Formula (IV), R^{2d} is hydrogen or optionally substituted C₁-C₆ alkyl. In some embodiments of a compound of Formula (IV), R^{2d} is hydrogen, C₁-C₆ alkyl, or C₁-C₆ haloalkyl. In some embodiments of a compound of Formula (IV), R^{2d} is hydrogen.

In some embodiments of a compound of Formula (IV), m is 1 or 2. In some embodiments of a compound of Formula (IV), m is 1. In some embodiments of a compound of Formula (IV), m is 2. In some embodiments of a compound of Formula (IV), m is 3. In some embodiments of a compound of Formula (IV), m is 4.

In some embodiments of a compound of Formula (IV), each R³¹ is independently hydrogen, halogen, —CN, —OR¹¹, —NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (IV), each R³¹ is independently hydrogen, halogen, —CN, —OH, or optionally substituted C₁-C₆ alkyl. In some embodiments of a compound of Formula (IV), each R³¹ is independently hydrogen, halogen, or optionally substituted C₁-C₆ alkyl. In some embodiments of a compound of Formula (IV), each R³¹ is independently hydrogen, halogen, C₁-C₆ alkyl, or C₁-C₆ haloalkyl. In some embodiments of a compound of Formula (IV), each R³¹ is hydrogen.

In some embodiments of a compound of Formula (IV), n3 is 2-4. In some embodiments of a compound of Formula (IV), n3 is 2. In some embodiments of a compound of Formula (IV), n3 is 3. In some embodiments of a compound of Formula (IV), n3 is 4.

Disclosed herein is a compound of Formula (V), or a pharmaceutically acceptable salt, solvate, or stereoisomer thereof:

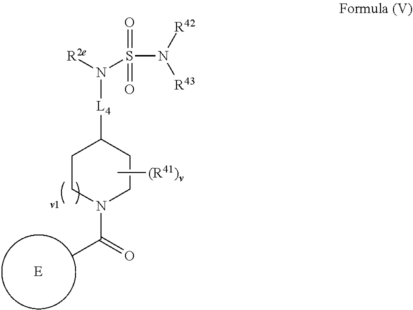

wherein
L₄ is —(CR⁴⁴R⁴⁵)ₙ₄—;
Ring E is optionally substituted cycloalkyl, optionally substituted heterocycloalkyl, optionally substituted aryl, or optionally substituted heteroaryl;
each R⁴¹ is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, and optionally substituted (C₁-C₆ alkyl)heteroaryl;
or two R⁴¹ on the same carbon are taken together to form an oxo;
R^{2e} is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R⁴² and R⁴³ are independently hydrogen, optionally substituted C₁-C₆ alkyl, optionally substituted cycloalkyl, optionally substituted heterocycloalkyl, optionally substituted aryl, or optionally substituted heteroaryl;
or R⁴² and R⁴³ taken together form an optionally substituted heterocycloalkyl;
each R⁴⁴ and R⁴⁵ are independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R⁴⁴ and R⁴⁵ on the same carbon are taken together to form an oxo;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
n4 is 1-4;
v is 1-4; and
v1 is 0 or 1.

In some embodiments of a compound of Formula (V), Ring E is optionally substituted cycloalkyl. In some embodiments of a compound of Formula (V), Ring E is optionally substituted cycloalkyl selected from cyclopropyl, cyclobutyl, cyclopentyl, and cyclohexyl.

In some embodiments of a compound of Formula (V), Ring E is optionally substituted aryl. In some embodiments of a compound of Formula (V), Ring E is optionally substituted phenyl.

In some embodiments of a compound of Formula (V), Ring E is optionally substituted heteroaryl. In some embodiments of a compound of Formula (V), Ring E is optionally substituted heteroaryl selected from quinolinyl, isoquinolinyl, quinazolinyl, naphthyridinyl, cinnolinyl, pyridopyridazinyl, phthalazinyl, indolyl, pyrrolopyridinyl, indazolyl, pyrazolopyridine, benzotriazolyl, benzimidazolyl, pyrrolopyrimidinyl, pyrazolopyrimidinyl, triazolopyrimidinyl, purinyl, pyrrolopyridinyl, pyrazolopyridinyl, triazolopyridinyl, and imidazopyridinyl. In some embodiments of a compound of Formula (V), Ring E is optionally substituted heteroaryl selected from 2-pyridinyl, 3-pyridinyl, 4-pyridimidyl, 5-pyridimidyl, and 2-pyrazinyl. In some embodiments of a compound of Formula (V), Ring E is heteroaryl optionally substituted with one, two, or three halogen, —CN, —OR¹¹, —NR¹¹R¹², —C(═O)OR¹¹, —C(═O)NR¹¹R¹², optionally substituted C₁-C₆ alkyl, optionally substituted C₁-C₆ heteroalkyl, optionally substituted cycloalkyl, optionally substituted heterocycloalkyl, optionally substituted aryl, or optionally substituted heteroaryl.

In some embodiments of a compound of Formula (V), Ring E is optionally substituted heterocycloalkyl. In some embodiments of a compound of Formula (V), Ring E is optionally substituted heterocycloalkyl selected from pyrrolidinyl, piperidinyl, piperazinyl, and morpholinyl. In some embodiments of a compound of Formula (V), Ring E is optionally substituted heterocycloalkyl selected from pyrrolidinyl, piperazinyl, and morpholinyl.

In some embodiments of a compound of Formula (V), Ring E is optionally substituted with one, two, or three halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl. In some embodiments of a compound of Formula (V), Ring E is optionally substituted with one, two, or three halogen, —CN, —OR¹¹, —NR¹¹R¹², —C(═O)OR¹¹, —C(═O)NR¹¹R¹², —NR¹¹C(═O)R¹⁰, optionally substituted C₁-C₆ alkyl, optionally substituted C₁-C₆ heteroalkyl, optionally substituted cycloalkyl, optionally substituted heterocycloalkyl, optionally substituted aryl, or optionally substituted heteroaryl. In some embodiments of a compound of Formula (V), Ring E is optionally substituted with one, two, or three halogen, —OR¹¹, —NR¹¹R¹², —NR¹¹C(═O)R¹⁰, or optionally substituted C₁-C₆ alkyl. In some embodiments of a compound of Formula (V), Ring E is optionally substituted with one, two, or three halogen, —OR¹¹, —NR¹¹R¹², —NR¹¹C(═O)R¹⁰, C₁-C₆ alkyl, or C₁-C₆ haloalkyl.

In some embodiments of a compound of Formula (V), R⁴² and R⁴³ are independently hydrogen or optionally substituted C₁-C₆ alkyl.

In some embodiments of a compound of Formula (V), R⁴² and R⁴³ taken together form an optionally substituted heterocycloalkyl. In some embodiments of a compound of Formula (V), R⁴² and R⁴³ taken together form an optionally substituted heterocycloalkyl selected from pyrrolidinyl, piperidinyl, piperazinyl, and morpholinyl.

In some embodiments of a compound of Formula (V), each R⁴⁴ and R⁴⁵ are independently hydrogen, halogen, —CN, —OR¹¹, —NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (V), each R⁴⁴ and R⁴⁵ are independently hydrogen, halogen, —CN, —OH, or optionally substituted C₁-C₆ alkyl. In some embodiments of a compound of Formula (V), each R⁴⁴ and R⁴⁵ are independently hydrogen, halogen, C₁-C₆ alkyl, or C₁-C₆ haloalkyl. In some embodiments of a compound of Formula (V), each R⁴⁴ and R⁴⁵ are independently hydrogen or halogen. In some embodiments of a compound of Formula (V), each R⁴⁴ and R⁴⁵ are hydrogen. In some embodiments of a compound of Formula (V), R⁴⁴ and R⁴⁵ on the same carbon are taken together to form an oxo.

In some embodiments of a compound of Formula (V), L₄ is —(CR⁴⁴R⁴⁵)ₙ₄—; n4 is 2 or 3; and each R⁴⁴ and R⁴⁵ are independently hydrogen or halogen.

In some embodiments of a compound of Formula (V), v1 is 0. In some embodiments of a compound of Formula (V), v1 is 1.

In some embodiments of a compound of Formula (V), R^{2e} is hydrogen or optionally substituted C₁-C₆ alkyl. In some embodiments of a compound of Formula (V), R^{2e} is hydrogen, C₁-C₆ alkyl, or C₁-C₆ haloalkyl. In some embodiments of a compound of Formula (V), R^{2e} is hydrogen.

In some embodiments of a compound of Formula (V), v is 1 or 2. In some embodiments of a compound of Formula (V), v is 1. In some embodiments of a compound of Formula (V), v is 2. In some embodiments of a compound of Formula (V), v is 3. In some embodiments of a compound of Formula (V), v is 4.

In some embodiments of a compound of Formula (V), each R⁴¹ is independently hydrogen, halogen, —CN, —OR¹¹, —NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (V), each R⁴¹ is independently hydrogen, halogen, —CN, —OH, or optionally substituted C₁-C₆ alkyl. In some embodiments of a compound of Formula (V), each R⁴¹ is independently hydrogen, halogen, or optionally substituted C₁-C₆ alkyl. In some embodiments of a compound of Formula (V), each R⁴¹ is independently hydrogen, halogen, C₁-C₆ alkyl, or C₁-C₆ haloalkyl. In some embodiments of a compound of Formula (V), each R⁴¹ is hydrogen.

In some embodiments of a compound of Formula (V), n4 is 2-4. In some embodiments of a compound of Formula (V), n4 is 2. In some embodiments of a compound of Formula (V), n4 is 3. In some embodiments of a compound of Formula (V), n4 is 4.

In some embodiments of a compound of Formula (I′), (I), (II), (III), (IV), or (V), R¹⁰ is optionally substituted C₁-C₆ alkyl, optionally substituted aryl, or optionally substituted heteroaryl. In some embodiments of a compound of Formula (I′), (I), (II), (III), (IV), or (V), R¹ is C₁-C₆ alkyl, C₁-C₆ haloalkyl, aryl, or heteroaryl.

In some embodiments of a compound of Formula (I′), (I), (II), (III), (IV), or (V), each R¹¹ and R¹² are each independently hydrogen, optionally substituted C₁-C₆ alkyl, optionally substituted aryl, or optionally substituted heteroaryl. In some embodiments of a compound of Formula (I′), (I), (II), (III), (IV), or (V), each R¹¹ and R¹² are each independently hydrogen, C₁-C₆ alkyl, C₁-C₆ haloalkyl, aryl, or heteroaryl.

In some embodiments of a compound of Formula (I′), (I), (II), (III), (IV), or (V), each R¹¹ is C₁-C₆ alkyl.

In some embodiments, the compound disclosed herein is selected from Table 1:

TABLE 1

| Ex | Name | Structure |
|---|---|---|
| 1 | N-(2-(1-(6,7-dimethoxy-2- (methylamino)quinazolin-4- yl)piperidin-4- yl)ethyl)methanesulfonamide | 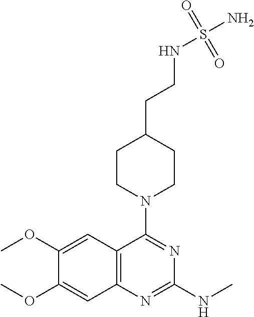 [SMILES: CNc1nc(N2CCC(CCNS(N)(=O)=O)CC2)c2cc(OC)c(OC)cc2n1] |
| 2 | 6,7-dimethoxy-4-(4-(2- (sulfamoylamino)ethyl)piperidin- 1-yl)quinoline-3-carboxamide | 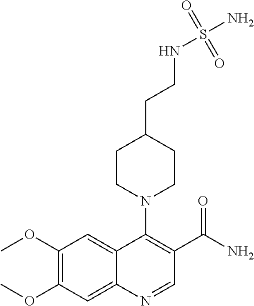 [SMILES: COc1cc2ncc(C(N)=O)c(N3CCC(CCNS(N)(=O)=O)CC3)c2cc1OC] |
| 3 | N-(2-(1-(1-methyl-1H- pyrazolo[3,4-d]pyrimidin-4- yl)piperidin-4-yl)ethyl)sulfonic amide | 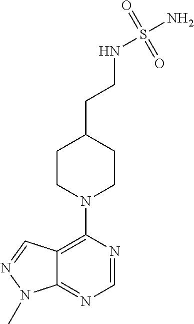 [SMILES: Cn1ncc2c(N3CCC(CCNS(N)(=O)=O)CC3)ncnc21] |
| 4 | N-(2-(6,7-dimethoxyquinazolin- 4-yl)-1,2,3,4- tetrahydroisoquinolin-5- yl)methanesulfonamide | 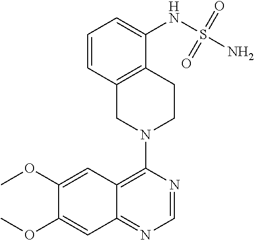 [SMILES: COc1cc2ncnc(N3CCc4c(cccc4NS(N)(=O)=O)C3)c2cc1OC] |
| 5 | 7-(6,7-dimethoxyquinazolin-4- yl)-2,7-diazaspiro[3.5]nonane-2- sulfonamide | 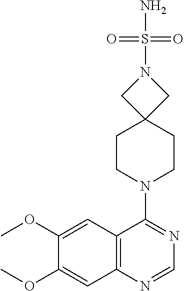 [SMILES: COc1cc2ncnc(N3CCC4(CC3)CN(S(N)(=O)=O)C4)c2cc1OC] |
| 6 | 2-(6,7-dimethoxyquinazolin-4- yl)-1,2,3,4- tetrahydroisoquinoline-5- sulfonamide | 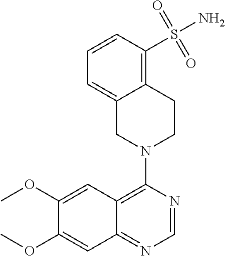 [SMILES: COc1cc2ncnc(N3CCc4c(cccc4S(N)(=O)=O)C3)c2cc1OC] |
| 7 | N-(3-(2-((6,7- dimethoxyquinazolin-4- yl)amino)ethyl)phenyl)sulfonic amide | 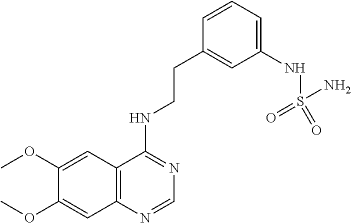 [SMILES: COc1cc2ncnc(NCCc3cccc(NS(N)(=O)=O)c3)c2cc1OC] |

In some embodiments, a compound described herein comprises a di-adenosine pentaphosphate analogue, an ATP analogue, an oxadiazole derivative, a biscoumarine derivative, or a combination. In some instances, an inhibitor of a 2′3′-cGAMP degradation polypeptide (e.g., a ENPP-1 inhibitor) comprises a compound, its analogue, or its derivative as illustrated in Scheme I.

### Scheme I.

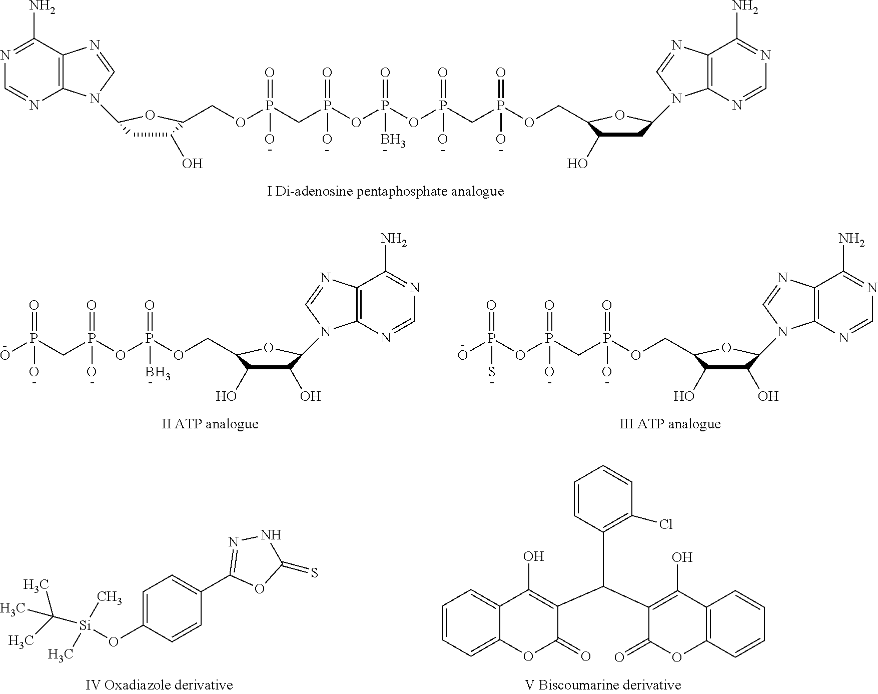

In some embodiments, a compound described herein comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141.

In some embodiments, a compound described herein is ARL67156:

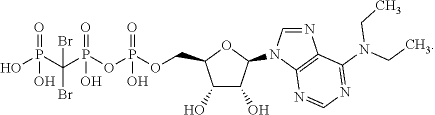 [SMILES: CCN(CC)c1ncnc2c1ncn2[C@@H]1O[C@H](COP(=O)(O)OP(=O)(O)C(Br)(Br)P(=O)(O)O)[C@@H](O)[C@H]1O]

In some embodiments, a compound described herein is diadenosine 5′,5″-boranopolyphosphonate:

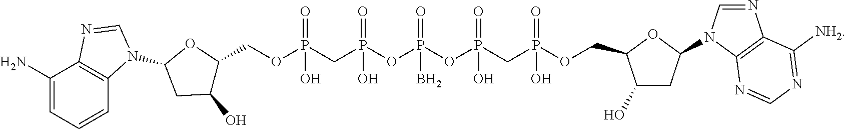

In some embodiments, a compound described herein is adenosine 5′-(α-borano)-β,γ-methylene triphosphate:

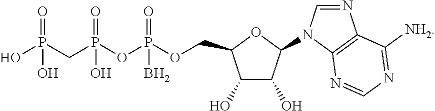

In some embodiments, a compound described herein is adenosine 5′-(γ-thio)-α,β-methylene triphosphate:

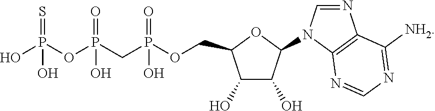 [SMILES: Nc1ncnc2c1ncn2[C@@H]1O[C@H](COP(=O)(O)CP(=O)(O)OP(O)(O)=S)[C@@H](O)[C@H]1O]

In some embodiments, a compound described herein is an oxadiazole derivative:

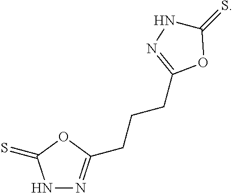 [SMILES: S=c1[nH]nc(CCCc2n[nH]c(=S)o2)o1]

In some embodiments, a compound described herein is a biscoumarine derivative:

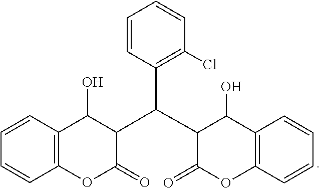 [SMILES: O=C1Oc2ccccc2C(O)C1C(c1ccccc1Cl)C1C(=O)Oc2ccccc2C1O]

In some embodiments, a compound described herein is reactive blue 2:

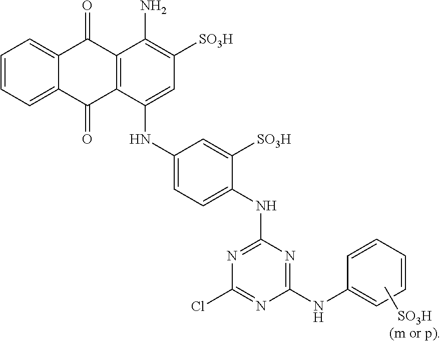

In some embodiments, a compound described herein is suramin:

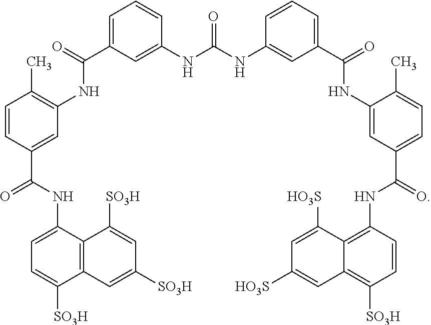 [SMILES: Cc1ccc(C(=O)Nc2ccc(S(=O)(=O)O)c3cc(S(=O)(=O)O)cc(S(=O)(=O)O)c23)cc1NC(=O)c1cccc(NC(=O)Nc2cccc(C(=O)Nc3cc(C(=O)Nc4ccc(S(=O)(=O)O)c5cc(S(=O)(=O)O)cc(S(=O)(=O)O)c45)ccc3C)c2)c1]

In some embodiments, a compound described herein is a quinazoline-4-piperidine-4-ethylsulfamide derivative:

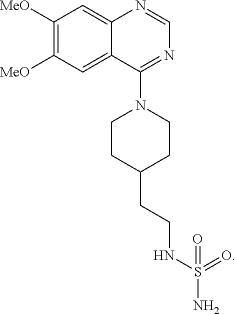 [SMILES: COc1cc2ncnc(N3CCC(CCNS(N)(=O)=O)CC3)c2cc1OC]

In some embodiments, a compound described herein is a thioacetamide derivative:

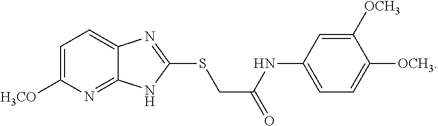 [SMILES: COc1ccc2nc(SCC(=O)Nc3ccc(OC)c(OC)c3)[nH]c2n1]

In some embodiments, a compound described herein is PSB-POM141: [symbol] (a Keggin-type inorganic complex).

In some embodiments, a compound described herein is 2-(3H-imidazo[4,5-b]pyridin-2-ylthio)-N-(3,4-dimethoxyphenyl)acetamide or a derivative, analog, or salt thereof.

In some embodiments, a compound described herein is 2-(6-Amino-9H-purin-8-ylthio)-N-(3,4-dimethoxyphenyl)-acetamide, or a salt thereof.

In some embodiments, a compound described herein is N-(3,4-Dimethoxyphenyl)-2-(5-methoxy-3H-imidazo[4,5-b]-pyridin-2-ylthio)acetamide or a salt thereof.

In some embodiments, a compound described herein is 2-(1-(6,7-Dimethoxyquinazolin-4-yl)piperidin-4-yl)ethyl sulfamide or a salt thereof.

In some embodiments, a compound described herein is ((1-(6,7-Dimethoxyquinazolin-4-yl)piperidin-4-yl)methyl)sulfamide or a salt thereof.

In some embodiments, a compound described herein is SK4A (SAT0037) or a derivative or salt thereof.

In some embodiments, a compound described herein is a PDE inhibitor described in Chang, et al., “Imidazopyridine- and purine-thioacetamide derivatives: potent inhibitors of nucleotide pyrophosphatase/phosphodiesterase I (NPP1),” J. of Med. Chem., 57:10080-10100 (2014).

In some embodiments, a compound described herein is a PDE inhibitor described in Lee, et al., “Thiazolo[3,2-α]benzimidazol-3(2H)-one derivatives: structure-activity relationships of selective nucleotide pyrophosphatase/phosphodiesterase1 (NPP1) inhibitors,” Bioorganic & Medicinal Chemistry, 24:3157-3165 (2016).

In some embodiments, a compound described herein is a PDE inhibitor described in Shayhidin, et al., “Quinazoline-4-piperidine sulfamides are specific inhibitors of human NPP1 and prevent pathological mineralization of valve interstitial cells,” British Journal of Pharmacology, 172:4189-4199 (2015).

In some embodiments, a compound described herein is a PDE inhibitor described in Li, et al., “Hydrolysis of 2′3′-cGAMP by ENPP-1 and design of nonhydrolyzable analogs,” Nature Chemical Biology, 10:1043-1048 (2014).

In some embodiments, a compound described herein is Compound 1:

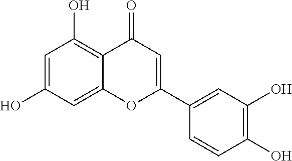 [SMILES: O=c1cc(-c2ccc(O)c(O)c2)oc2cc(O)cc(O)c12]

or a derivative, analog, or salt thereof.

In some embodiments, a compound described herein is Compound 2:

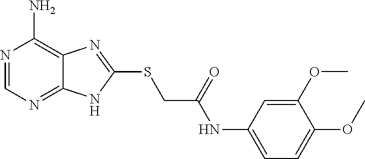 [SMILES: COc1ccc(NC(=O)CSc2nc3c(N)ncnc3[nH]2)cc1OC]

or a derivative, analog, or salt thereof.

In some embodiments, a compound described herein is Compound 3:

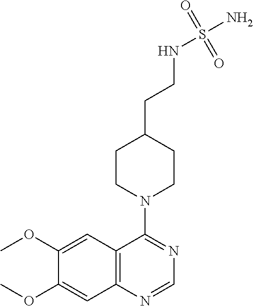 [SMILES: COc1cc2ncnc(N3CCC(CCNS(N)(=O)=O)CC3)c2cc1OC]

or a derivative, analog, or salt thereof.

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the metabolites, pharmaceutically acceptable salts, esters, prodrugs, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, or stereoisomer thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran, or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Compound Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Alkyl” refers to an optionally substituted straight-chain, or optionally substituted branched-chain saturated hydrocarbon monoradical having from one to about ten carbon atoms, or from one to six carbon atoms, wherein a sp3-hybridized carbon of the alkyl residue is attached to the rest of the molecule by a single bond. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms, wherein an sp2-hybridized carbon of the alkenyl residue is attached to the rest of the molecule by a single bond. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted as described below, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted as described below, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, phosphorous and sulfur. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized. In some embodiments, the heterocycloalkyl is a 3- to 6-membered heterocycloalkyl. In some embodiments, the heterocycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted as described below, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroalkyl” refers to an alkyl group in which one or more skeletal atoms of the alkyl are selected from an atom other than carbon, e.g., oxygen, nitrogen (e.g. —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl. Unless stated otherwise specifically in the specification, a Heteroalkyl is optionally substituted as described below, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted as described below, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests. For example, the term “treat” or “treating” with respect to tumor cells refers to stopping the progression of said cells, slowing down growth, inducing regression, or amelioration of symptoms associated with the presence of said cells.

The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include a synthetic molecule described supra. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, “derivative” refers to a chemically or biologically modified version of a chemical compound that is structurally similar to a parent compound and (actually or theoretically) derivable from that parent compound. In some cases, a derivative has different chemical or physical properties relative to the parent compound. For example, the derivative may be more hydrophilic or it may have altered reactivity as compared to the parent compound. Derivatization (i.e., modification) may involve substitution of one or more moieties within the molecule (e.g., a change in functional group) that do not substantially alter the function of the molecule for a desired purpose. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds which simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

As used herein, “analogue” refers to a chemical compound that is structurally similar to another but differs slightly in composition (as in the replacement of one atom by an atom of a different element or in the presence of a particular functional group), but may or may not be derivable from the parent compound. A “derivative” differs from an “analogue” in that a parent compound may be the starting material to generate a “derivative,” whereas the parent compound may not necessarily be used as the starting material to generate an “analogue.”

### EXAMPLES

These examples are provided for illustrative purposes only and not to limit the scope of the claims provided herein.

### Example 1

Co-Crystal Structure of Human ENPP1

ENPP1 Expression, Crystallization, and Data Collection

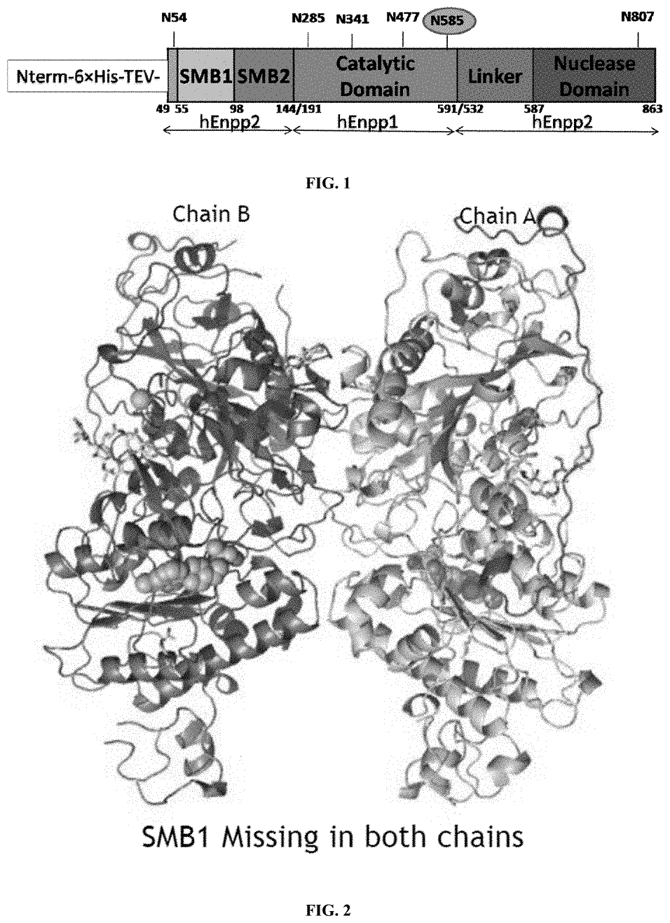

An exemplary chimeric human ENPP1 (hENPP1) construct used in this example (also see FIG. 1):
Nterm-HisTag-TEV-hENPP2(aa49-144)-hENPP1(aa191-591)-hENPP2(aa523-863)

A chimeric hENPP1 protein encoded by the chimeric hENPP1 construct described above was expressed and purified in an insect cell system and was subsequently purified by size exclusion on a Superdex 200 PG column.

The chimeric hENPP1 protein at a concentration of 7 mg/mL was incubated with Compound 3 at a 1:10 (protein:compound) molar ratio for about 3 hours at 4° C. prior to crystallization setup. The chimeric hENPP1-Compound 3 complex was crystallized in 0.1M HEPES pH 7.2, 22% (w/v) PEG 4000, and 10% (w/v) isopropanol. The crystals were grown by the sitting-drop vapor diffusion method at 22° C. Crystals were flash frozen in liquid nitrogen and cryoprotected with the respective reservoir solution supplemented with about 12% glycerol.

Structure Determination and Refinement

Diffraction data were collected on the MX2 beamline at the Australian Synchrotron, part of ANSTO. Data were processed with CCP4 Program Suite: AIMLESS.

Table 2 illustrates the data collection statistics.

Data Collection Statistics

| Number of frames | | 1800 |
|---|---|---|
| Oscillation width (°) | | 0.1 |
| Exposure per frame (sec) | | 0.01 |
| Space Group | | P2₁2₁2₁ |
| Resolution Range (Å) | | 47.00 - 2.75 (2.84-2.75) |
| Unit Cell constants | (A) | a = 76.41; b = 119.21; c = 201.56 |
| | (°) | A = β = γ = 90.0 |

| | |
|---|---|
| R_{merge} (%) | 10.3 (63.8) |
| Rpim (%) | 4.3 (26.7) |
| CC_{1/2} | 0.998 (0.845) |
| Completeness (%) | 99.4 (94.5) |
| <I>/σ(<I>) | 11.6 (2.1) |
| Average redundancy | 6.8 (6.5) |

Table 3 illustrates the refinement statistics.

Refinement Statistics

| No of molecules in | | 2 |
|---|---|---|
| asymmetric unit | | |
| Start model PDB-ID | | 4GTW and 4ZG6 |
| N-terminal amino acid build | | Chain A - 146 (Gly) |
| in structure (hENPP1) | | Chain B - 147 (Trp) |
| C-terminal amino acid build | | Chain A - 923 (Ile) |
| in structure (hENPP1) | | Chain B - 923 (Ile) |
| Final R-factor (Rfree) | | 0.207 (0.260) |
| Ramachandran | Core | 87.7 |
| Statistics (%) | | |
| | Allowed | 11.9 |
| | Generous | 0.2 |
| | Disallowed | 0.2 |

| | |
|---|---|
| No of water molecules | 80 |
| Ligand bound status | Yes in both chains A and B |
| Number of metal atoms | 2 Zn²⁺ ions and 1 Ca²⁺ ion in |
| | each chain |

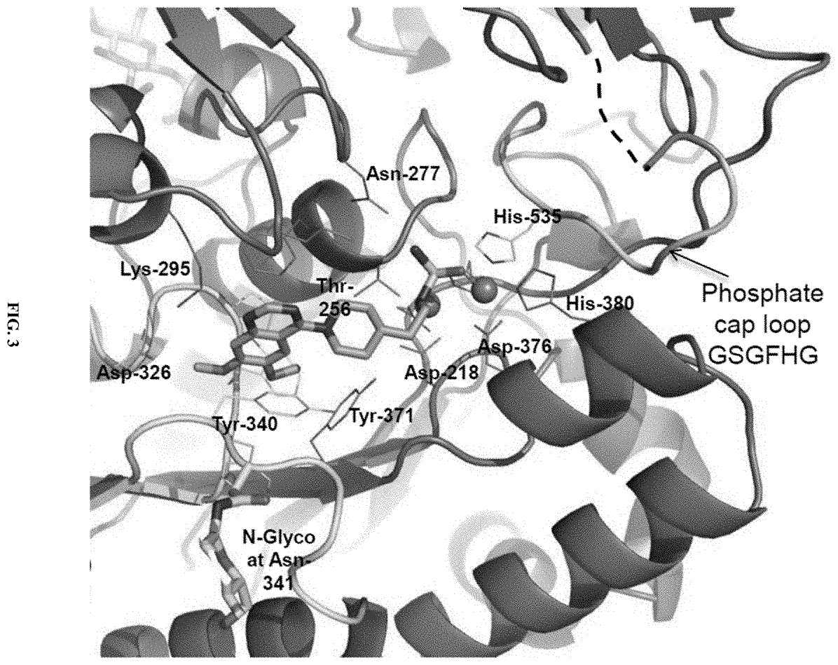

FIG. 2 illustrates the crystal structure of the hENPP1 in complex with Compound 3. The catalytic domain of hENPP1 is shown in green.

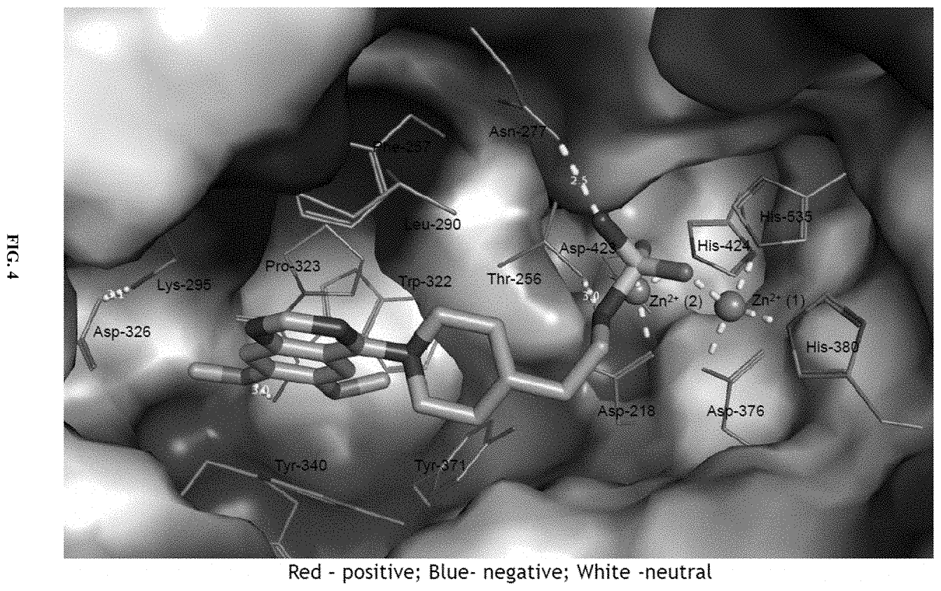

FIG. 3 and FIG. 4 illustrate close-up views of Compound 3 within the catalytic pocket in two different orientations.

As observed in the crystal structure, Phe257 and Tyr340 formed Pi-Pi stacking interaction with the quinozoline moiety of Compound 3. The dimethoxy group of Compound 3 was observed to point toward the loop region of Phe321-Pro323. Thr256 was observed to interact with the sulfonamide moiety. The two Zinc ions were observed to interact with the oxygen atom of the sulfonamide moiety. Asn277 was observed to interact with the NH₂ group of the sulfonamide moiety. Tyr-371 was observed to be in close proximity to the piperidine group of Compound 3.

### Example 2

ENPP1 Enzyme Assay with cGAMP Substrate:

Ectonucleotide pyrophosphatase/phosphodiesterase 1 (ENPP-1) is a transmembrane glycoprotein that hydrolyzes nucleotides and nucleotide derivatives with the formation of nucleoside-5′-monophosphates. ENPP-1 hydrolyzes 2′3′-cGAMP (cGAMP), breaking it down into 5′-AMP and 5′-GMP. The 5′-AMP formed from the reaction is detected using the AMP-Glo® Kit (Promega). The assay kit contains two reagents. The first reagent terminates the enzymatic reaction, removes ATP (using adenylyl cyclase), and converts 5′-AMP produced into ADP (using polyphosphate: AMP phosphotransferase). The second reagent converts ADP to ATP (using adenylate kinase) and generates light from ATP using the luciferin/luciferase reaction. The amount of light measured is proportional to the amount of 5′-AMP produced by ENPP1.

Different concentrations of exemplary ENPP1 inhibitors (synthetic molecules #1-7; see Table 1) were pre-incubated with 5 ng/well of human ENPP-1 enzyme (R&D Systems) for 15 minutes at 37° C. The reaction was initiated by adding 20 μM 2′3′-cGAMP and incubating for 30 minutes at 37° C. The final assay reaction mixture contained a buffer of 50 mM Tris pH 8.0, 250 mM NaCl, 0.5 mM CaCl₂, 1 μM ZnCl₂ and 1% DMSO. At the end of the incubation, the reaction was stopped by adding 12 μl of AMP-Glo reagent-1 and mixing the reaction uniformly for 5 minutes, followed by incubation at room temperature for one hour. Then 25 μl of AMP Glo reagent-2 was added to the reaction, mixed uniformly with a pipette, and incubated at room temperature for one hour to convert the ADP formed from reagent-1 to ATP and light. The generated light was measured in a Perkin Elmer Victor® instrument. Maximal activity control samples (containing enzyme, substrate, and buffer in the absence of ENPP1 inhibitors: MAX) and background control samples (containing enzyme, substrate, and buffer plus a fully inhibitory concentration (3 μM) of the reference ENPP1 Inhibitor, MV-0000002: MIN) were simultaneously evaluated in order to calculate the percent inhibition at each compound concentration as follows:

% inhibition=(([MAX−MIN]−[COMPOUND−MIN])/[MAX−MIN])*100

The IC₅₀ values for percent inhibition versus compound concentration were determined by fitting the inhibition curves using a four-parameter variable slope model in GraphPad Prism® software. Ki values are derived from the IC₅₀ values using the Cheng-Prusoff equation:

Ki=IC₅₀/(1+[cGAMP]/Km),

where routinely [cGAMP]=20 μM and Km=16 μM

ENPP1 Enzyme Assay with TMP-pNP Substrate:

ENPP-1 hydrolyzes thymidine 5′monophosphate p-nitrophenyl ester (TMP-pNP) to nucleotide-5′-monophosphate and p-nitrophenol, which is a chromogenic product. The amount of p-nitrophenol product formed is measured using its absorbance at 405 nm, which is directly proportional to enzyme activity. Different concentrations of inhibitors (synthetic molecules #1-7; see Table 1) were pre-incubated with 15 ng/well of human ENPP-1 enzyme (R&D Systems) for 15 minutes at 37° C. The reaction was initiated by adding 200 μM TMP-pNP and incubating for 10 minutes at 37° C. The final assay reaction mixture contained a buffer of 50 mM Tris pH 8.0, 250 mM NaCl, 0.5 mM CaCl₂, 1 μM ZnCl₂ and 1% DMSO. The amount of product formed was measured directly ina Tecan® spectrophotometer. Maximal activity control samples (containing enzyme, substrate, and buffer in the absence of ENPP1 inhibitors: MAX) and background control samples (containing enzyme, substrate, and buffer plus a fully inhibitory concentration (3 μM) of the reference ENPP1 Inhibitor, MV-0000002: MIN) are simultaneously evaluated in order to calculate the percent inhibition at each compound concentration as follows:

% inhibition=(([MAX−MIN]−[COMPOUND−MIN])/[MAX−MIN])*100

The IC₅₀ values for percent inhibition versus compound concentration were determined by fitting the inhibition curves (percent inhibition versus inhibitor concentration) using a four-parameter variable slope model in GraphPad Prism® software. Ki values are derived from the IC₅₀ values using the Cheng-Prusoff equation:

Ki=IC₅₀/(1+[TMP-pNP]/Km),

where routinely [TMP-pNP]=200 μM and Km=151 μM

The data is shown in Table 4.

| | cGAMP: | cGAMP: | | TMP-pNP: | |
|---|---|---|---|---|---|
| | % | % | | % | |
| | Inhibition | Inhibition | cGAMP: | inhibition | TMP-pNP: |
| Ex. | at 1 uM | at 10 uM | Ki (nM) | at 10 uM | Ki (nM) |
| 1 | A | | | A | |
| 2 | A | | | A | |
| 3 | | A | *** | | *** |
| 4 | | | * | | ** |
| 5 | | A | * | A | ** |
| 6 | A | | ** | | *** |
| 7 | | A | ** | | *** |

% inhibition: A ≥ 75%; 75% > B ≥ 50%; 50% > C ≥ 25%; and 25% > D.
Ki: * ≤ 100 nm; 100 nm < ** ≤ 1 μm; and 1 μm < ***.
NA = Not active.

### Example 3

Table 5 illustrates an exemplary ENPP1 sequence.

| Name | Sequence | SEQ ID NO: |
|---|---|---|
| ENPP1 | MERDGCAGGGSRGGEGGRAPREGPAGNGRDRGRSHAAEAPG | 1 |
| (homo sapiens) | DPQAAASLLAPMDVGEEPLEKAARARTAKDPNTYKVLSLVLS | |
| (NCBI | VCVLTTILGCIFGLKPSCAKEVKSCKGRCFERTFGNCRCDAAC | |
| Accession No.: | VELGNCCLDYQETCIEPEHIWTCNKFRCGEKRLTRSLCACSDD | |
| NP_006199.2) | CKDKGDCCINYSSVCQGEKSWVEEPCESINEPQCPAGFETPPTL | |
| | LFSLDGFRAEYLHTWGGLLPVISKLKKCGTYTKNMRPVYPTKT | |
| | FPNHYSIVTGLYPESHGIIDNKMYDPKMNASFSLKSKEKFNPE | |
| | WYKGEPIWVTAKYQGLKSGTFFWPGSDVEINGIFPDIYKMYNG | |
| | SVPFEERILAVLQWLQLPKDERPHFYTLYLEEPDSSGHSYGPVS | |
| | SEVIKALQRVDGMVGMLMDGLKELNLHRCLNLILISDHGMEQ | |
| | GSCKKYIYLNKYLGDVKNIKVIYGPAARLRPSDVPDKYYSFNY | |
| | EGIARNLSCREPNQHFKPYLKHFLPKRLHFAKSDRIEPLTFYLD | |
| | PQWQLALNPSERKYCGSGFHGSDNVFSNMQALFVGYGPGFKH | |
| | GIEADTFENIEVYNLMCDLLNLTPAPNNGTHGSLNHLLKNPVY | |
| | TPKHPKEVHPLVQCPFTRNPRDNLGCSCNPSILPIEDFQTQFNLT | |
| | VAEEKIIKHETLPYGRPRVLQKENTICLLSQHQFMSGYSQDILM | |
| | PLWTSYTVDRNDSFSTEDFSNCLYQDFRIPLSPVHKCSFYKNNT | |
| | KVSYGFLSPPQLNKNSSGIYSEALLTTNIVPMYQSFQVIWRYFH | |
| | DTLLRKYAEERNGVNVVSGPVFDFDYDGRCDSLENLRQKRRV | |
| | IRNQEILIPTHFFIVLTSCKDTSQTPLHCENLDTLAFILPHRTDNS | |
| | ESCVHGKHDSSWVEELLMLHRARITDVEHITGLSFYQQRKEPV | |
| | SDILKLKTHLPTFSQED | |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. An ecto-nucleotide pyrophosphatase/phosphodiesterase (ENPP) complex having a synthetic molecule in contact with at least one residue at an amino acid position corresponding to amino acid residues D218, T256, F257, N277, L290, K295, W322, P323, D326, Y340, Y371, D376, H380, D423, H424, or H535 as set forth in SEQ ID NO: 1, wherein the synthetic molecule is not a hydrolysis product of a nucleoside triphosphate.

2. The ENPP-molecule complex of claim 1, wherein the molecule is in contact with at least one residue at an amino acid position corresponding to:
amino acid residues D218, T256, F257, N277, W322, D326, Y340, D376, H380, D423, H424, or H535 as set forth in SEQ ID NO: 1;
amino acid residues T256, F257, N277, W322, P323, D326, Y340, or Y371 as set forth in SEQ ID NO: 1;
amino acid residues T256, F257, N277, W322, P323, Y340, or Y371 as set forth in SEQ ID NO: 1;
amino acid residues F257, W322, D326, or Y340 as set forth in SEQ ID NO: 1;
amino acid residues F257 or Y340 as set forth in SEQ ID NO: 1; or
amino acid residues D326 or W322 as set forth in SEQ ID NO: 1.

3. The ENPP-molecule complex of claim 1, wherein the synthetic molecule has a structure represented by Formula (I), or a pharmaceutically acceptable salt, solvate, or stereoisomer thereof: 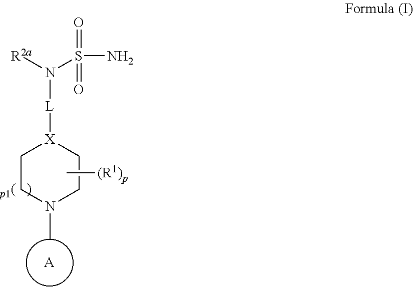
wherein
L is —(CR³R⁴)ₙ—;
X is —N— or —CH—;
Ring A is
(a) an optionally substituted heteroaryl that is not quinazolinyl or pyrimidyl; or
(b) an optionally substituted heterocycloalkyl; or
(c) a ring selected from 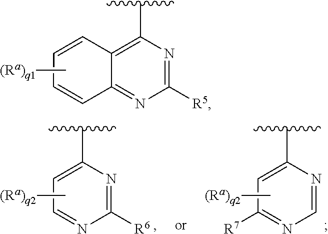
each R¹ is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, and optionally substituted (C₁-C₆ alkyl)heteroaryl;
or two R¹ on the same carbon are taken together to form an oxo;
R^{2a} is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R³ and R⁴ are independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R³ and R⁴ on the same carbon are taken together to form an oxo;
R⁵ is halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ heteroalkyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R⁶ is hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl; provided that R⁶ is not substituted imidazolyl;
R⁷ is hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted (C₁-C₆ alkyl)aryl, optionally substituted heteroaryl, or optionally substituted (C₁-C₆ alkyl)heteroaryl; provided that R⁷ is not substituted imidazolyl;
each R^{a} is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
n is 1-4;
p is 1-4;
p1 is 0 or 1;
q1 is 1-4; and
q2 is 1-2.

4. The ENPP-molecule complex of claim 3, wherein R^{2a} is hydrogen.

5. The ENPP-molecule complex of claim 3, wherein: L is —(CR³R⁴)ₙ—; n is 2; and each R³ and R⁴ are independently hydrogen or halogen.

6. The ENPP-molecule complex of claim 3, wherein X is —CH—.

7. The ENPP-molecule complex of claim 3, wherein X is —N—.

8. The ENPP-molecule complex of claim 3, wherein Ring A is selected from: 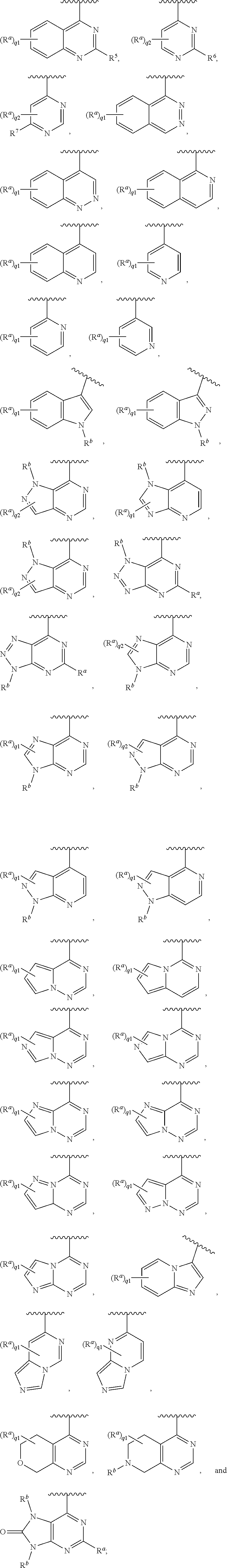
and
each R^{b} is independently hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl.

9. The ENPP-molecule complex of claim 3, wherein Ring A is

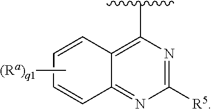

10. The ENPP-molecule complex of claim 9, wherein each R^{a} is independently hydrogen, halogen, —CN, —OR¹¹, optionally substituted C₁-C₆ alkyl, or optionally substituted C₁-C₆ heteroalkyl; and q1 is 2 or 3.

11. The ENPP-molecule complex of claim 9, wherein R⁵ is halogen, —CN, —OR¹¹, —NR¹¹R¹², —C(═O)OR¹¹, —NR¹¹C(═O)R¹⁰, optionally substituted C₁-C₆ heteroalkyl, optionally substituted cycloalkyl, optionally substituted heterocycloalkyl, optionally substituted aryl, or optionally substituted heteroaryl.

12. The ENPP-molecule complex of claim 3, wherein Ring A is

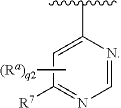

13. The ENPP-molecule complex of claim 12, wherein each R^{a} is independently hydrogen, halogen, —CN, —OR¹¹, optionally substituted C₁-C₆ alkyl, or optionally substituted C₁-C₆ heteroalkyl; and q2 is 1.

14. The ENPP-molecule complex of claim 12, wherein R⁷ is hydrogen, halogen, —CN, —OR¹¹, —NR¹¹R¹², —NR¹¹C(═O)R¹⁰, —C(═O)NR¹¹R¹², optionally substituted C₁-C₆ alkyl, optionally substituted C₁-C₆ heteroalkyl, optionally substituted cycloalkyl, optionally substituted heterocycloalkyl, optionally substituted aryl, or optionally substituted heteroaryl; provided that R is not substituted imidazolyl.

15. The ENPP-molecule complex of claim 3, wherein Ring A is

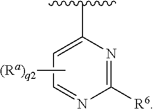

16. The ENPP-molecule complex of claim 15, wherein each R^{a} is independently hydrogen, halogen, —CN, —OR¹¹, optionally substituted C₁-C₆ alkyl, or optionally substituted C₁-C₆ heteroalkyl; and q2 is 1.

17. The ENPP-molecule complex of claim 15, wherein R⁶ is hydrogen, halogen, —CN, —OR¹¹, —NR¹¹R¹², —NR¹¹C(═O)R¹⁰, optionally substituted C₁-C₆ alkyl, optionally substituted C₁-C₆ heteroalkyl, optionally substituted cycloalkyl, optionally substituted heterocycloalkyl, optionally substituted aryl, or optionally substituted heteroaryl; provided that R⁶ is not substituted imidazolyl.

18. The ENPP-molecule complex of claim 1, wherein the synthetic molecule has a structure represented by Formula (III), or a pharmaceutically acceptable salt, solvate, or stereoisomer thereof: 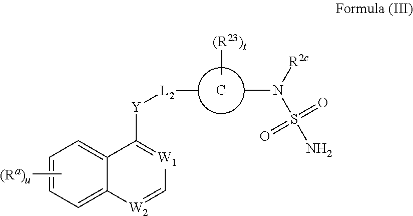
wherein
Y is —O— or —NR²⁰—;
L₂ is a bond or —(CR²¹R²²)ₙ₂—;
W₁ and W₂ are independently N or CR^{a}; provided that at least one of W₁ or W₂ is N;
Ring C is aryl, heteroaryl, cycloalkyl, or heterocycloalkyl;
each R²³ is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R^{2c} is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R²⁰ is hydrogen, —SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R²¹ and R²² are independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R²¹ and R²² on the same carbon are taken together to form an oxo;
each R^{a} is independently hydrogen, halogen, —CN, —OR¹¹, —SR¹¹, —S(═O)R¹⁰, —NO₂, —NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
t is 1-4;
n2 is 1 or 2; and
u is 1-4.

19. The ENPP-molecule complex of claim 18, wherein W₁ and W₂ are N.

20. The ENPP-molecule complex of claim 18, each R^{a} is independently hydrogen, halogen, —CN, —OR¹¹, —NR¹¹R¹², —C(═O)OR¹¹, —OC(═O)OR¹¹, —C(═O)NR¹¹R¹², optionally substituted C₁-C₆ alkyl, optionally substituted C₁-C₆ heteroalkyl, optionally substituted cycloalkyl, or optionally substituted heterocycloalkyl; and u is 1-3.

21. The ENPP-molecule complex of claim 18, wherein each R²³ is independently hydrogen, halogen, or optionally substituted C₁-C₆ alkyl.

22. The ENPP-molecule complex of claim 18, wherein L₂ is a bond.

23. The ENPP-molecule complex of claim 18, wherein L₂ is —(CR²¹R²²)ₙ₂—; n2 is 1 or 2; and
each R²¹ and R²² are independently hydrogen or halogen

24. The ENPP-molecule complex of claim 18, wherein R^{2c} is hydrogen.

25. The ENPP-molecule complex of claim 18, wherein Ring C is a 6-membered aryl.

26. The ENPP-molecule complex of claim 16, wherein Ring C is a 5- or 6-membered heteroaryl.

27. The ENPP-molecule complex of claim 1, wherein the contact comprises covalent interaction, non-covalent interaction, or a combination thereof.

28. The ENPP-molecule complex of claim 1, wherein the contact comprises hydrogen bonding, hydrophobic interaction, ionic interaction, Van der Waals interaction, electrostatic interaction, pi bonding, or a combination thereof.

29. The ENPP-molecule complex of claim 1, wherein the ecto-nucleotide pyrophosphatase/phosphodiesterase is ecto-nucleotide pyrophosphatase/phosphodiesterase 1 (ENPP1).

30. The ENPP-molecule complex of claim 1, wherein the hydrolysis product is AMP, TMP, GMP, or CMP.
